(19) United States
(12) Patent Application Publication
(10) Pub. No.: US 2003/0134413 A1
(43) Pub. Date: Jul. 17, 2003

(54) Cell production

(76) Inventors: Peter David Rathjen, Mircham (AU); Joy Rathjen, Mircham (AU)

Correspondence Address: SUTHERLAND ASBILL & BRENNAN LLP, 999 PEACHTREE STREET, N.E., ATLANTA, GA, 30309, US

(21) Appl. No.: 10/181,359

(22) PCT Filed: Jan. 12, 2001
(86) PCT No.: PCT/AU01/00030

(30) Foreign Application Priority Data
Jan. 14, 2000 (AU) PQ 5098
Apr. 20, 2000 (AU) PQ 7045

(51) Int. Cl. C12N 5/08

(52) U.S. Cl. 435/368

(57) ABSTRACT

A method of producing neurectoderm cells, which method includes providing a source of early primitive ectoderm-like (EPL) cells; a conditioned medium as hereinbefore defined; or an extract therefrom exhibiting neural inducing properties; and contacting the EPL cells with the conditioned medium, for a time sufficient to generate controlled differentiation to neurectoderm cells.

The present invention relates to neurectoderm cells and to differentiated or partially differentiated cells derived therefrom. The present invention also relates to methods of producing, differentiating and culturing the cells of the invention, and to uses thereof.

Initial developmental events within the mammalian embryo entail the elaboration of extra-embryonic cell lineages and result in the formation of the blastocyst, which comprises trophectoderm, primitive endoderm and a pool of pluripotent cells, the inner cell mass (ICM/epiblast). As development continues, the cells of the ICM/epiblast undergo rapid proliferation, selective apoptosis, differentiation and reorganisation as they develop to form the primitive ectoderm. In the mouse, the cells of the ICM begin to proliferate rapidly around the time of blastocyst implantation. The resulting pluripotent cell mass expands into the blastocoelic cavity. Between 5.0 and 5.5 dpc (days post coitus) the inner cells of the epiblast undergo apoptosis to form the proamniotic cavity. The outer, surviving cells, or early primitive ectoderm, continue to proliferate and by 6.0-6.5 dpc have formed a pseudo-stratified epithelial layer of pluripotent cells, termed the primitive or embryonic ectoderm. Primitive ectoderm cells are pluripotent, and distinct from cells of the ICM in terms of morphology, gene expression and differentiation potential.

By 4.5 dpc pluripotent cells exposed to the blastocoelic cavity have differentiated to form primitive endoderm. The primitive endoderm gives rise to two distinct endodermal cell populations, visceral endoderm, which remains in contact with the epiblast, and parietal endoderm, which migrates away from the pluripotent cells to form a layer of endoderm adjacent to the trophectoderm. Formation of these endodermal layers is coincident with formation of primitive ectoderm and creation of an inner cavity. Visceral endoderm is known to express signals that influence pluripotent cell differentiation.

At gastrulation pluripotent cells of the primitive ectoderm differentiate to form the three germ layers of the embryo: mesoderm, endoderm and ectoderm. Pluripotent cells from this time are confined to the germline. Differentiation of primitive ectoderm cells in the distal and anterior regions of the embryo is directed along the ectodermal lineage forming definitive ectoderm, a transient embryonic cell type fated to form neurectoderm and surface ectoderm.

Neurectoderm cells are found in the mammalian embryo in the neural plate, which folds and closes to form the neural tube. These cells are the precursors to all neural lineages. They have the capacity to differentiate into all neural cell types present in the central nervous system (CNS) and peripheral nervous system (PNS). In the CNS these cells include multiple neuron subtypes and glia (eg; astrocytes and oligodendrocytes). Neural cells of the peripheral nervous system also include many different types of neurons and glial cells. Peripheral neural cells differentiate from transient embryonic precursor cells termed neural crest cells, which arise from the neural tube. Neural crest cells are also precursor cells to non-neural cells, including melanocytes, cartilage and connective tissue of the head and neck, and cells of cardiac outflow septation (Anderson, 1989).

In the human and in other mammals, formation of the blastocyst, including development of ICM cells and their progression to pluripotent cells of the primitive ectoderm, and subsequent differentiation to form the embryonic germ layers and differentiated cells, follow a similar developmental process.

Pluripotent cells can be isolated from the preimplantation mouse embryo as embryonic stem (ES) cells. ES cells can be maintained indefinitely as a pluripotent cell population in vitro, and, when reintroduced into a host blastocyst, can contribute to all adult tissues of the mouse including the germ cells. ES cells, therefore, retain the ability to respond to all the signals that regulate normal mouse development. EPL cells are a separate population of pluripotent cells distinct from ES cells. EPL cells are equivalent to early primitive ectoderm cells of the post-implantation embryo, and can be maintained, proliferated and differentiated in a controlled manner in vitro. EPL cells and their properties are described in International patent application WO99/53021.

ES cells and EPL cells represent powerful model systems for the investigation of mechanisms underlying pluripotent cell biology and differentiation within the early embryo, as well as providing opportunities for embryo manipulation and resultant commercial, medical and agricultural applications. Furthermore, appropriate proliferation and differentiation of ES and EPL cells can be used to generate an unlimited source of cells suited to transplantation for treatment of diseases which result from cell damage or dysfunction.

Other pluripotent cells and cell lines including in vivo or in vitro derived ICM/epiblast, in vivo or in vitro derived primitive ectoderm, primordial germ cells (EG cells), teratocarcinoma cells (EC cells), and pluripotent cells derived by dedifferentiation or by nuclear transfer will share some or all of these properties and applications.

The successful isolation, long term clonal maintenance, genetic manipulation and germ-line transmission of pluripotent cells from species other than rodents has generally been difficult to date and the reasons for this are unknown. International patent application WO97/32033 and U.S. Pat. No. 5,453,357 describe pluripotent cells including cells from species other than rodents. Primate ES cells have been described in International patent application WO96/23362, and in U.S. Pat. No. 5,843,780, and human EG cells have been described in International patent application WO98/43679.

The differentiation of murine ES cells can be regulated in vitro by the cytokine leukaemia inhibitory factor (LIF) and other gp130 agonists or by culture on feeder cells which promote self-renewal and prevent differentiation of the stem cells. Differentiation in vitro of human ES cells is not inhibited by LIF, but is inhibited by culture on feeder cells.

The ability to form predominantly homogeneous populations of partially differentiated or terminally differentiated cells by differentiation in vitro of pluripotent cells has proved problematic. Current approaches involve the formation of embryoid bodies from pluripotent cells, in a manner that is not controlled and does not result in homogeneous populations. Mixed cell populations such as those in embryoid bodies of this type are generally unlikely to be suitable for therapeutic or commercial use.

Selection procedures have been used to obtain cell populations enriched in neural cells from embryoid bodies. These include manipulation of culture conditions to select for neural cells (Okabe et al, 1996), and genetic modification of ES cells to allow selection of neural cells by antibiotic resistance (Li et al, 1998).

In these procedures the differentiation of pluripotent cells in vitro does not involve biological molecules that direct differentiation in a controlled manner. Hence homogeneous synchronous, populations of neurectoderm cells with unrestricted neural differentiation capability are not produced, restricting the ability to derive essentially homogeneous populations of partially differentiated or differentiated neural cells.

Chemical inducers such as retinoic acid have also been used to form neural lineages from a variety of pluripotent cells including ES cells (Bain et al, 1995). However the route of retinoic acid-induced neural differentiation has not been well characterised, and the repetoire of neural cell types produced appears to be generally restricted to ventral somatic motor, branchiomotor or visceromotor neurons (Renoncourt et al, 1998).

In summary it has not been possible to control the differentiation of pluripotent cells in vitro, to provide homogeneous, synchronous populations of neurectoderm cells with unrestricted neural differentiation capacity. Similarly methods have not been developed for the derivation of neurectoderm cells from pluripotent cells, in a manner that parallels their formation during embryogenesis. These limitations have restricted the ability to form essentially homogeneous, synchronous populations of partially differentiated and terminally differentiated neural cells in vitro, and have restricted their further development for therapeutic and commercial applications.

Neural stem cells and precursor cells have also been derived from foetal brain and adult primary central nervous system tissue in a number of species, including rodent and human (e.g. see U.S. Pat. No. 5,753,506 (Johe), U.S. Pat. No. 5,766,948 (Gage), U.S. Pat. No. 5,589,376 (Anderson and Stemple), U.S. Pat. No. 5,851,832 (Weiss et al), U.S. Pat. No. 5,958,767 (Snyder et al) and U.S. Pat. No. 5,968,829 (Carpenter). However, each of these disclosures fails to describe a predominantly homogeneous population of neural stem cells able to differentiate into all neural cell types of the central and peripheral nervous systems, and/or essentially homogeneous populations of partially differentiated or terminally differentiated neural cells derived from neural stem cells by controlled differentiation.

Furthermore, it is not clear whether cells derived from primary foetal or adult tissue can be expanded sufficiently to meet potential cell and gene therapy demands.

It is an object of the present invention to overcome, or at least alleviate, one or more of the difficulties or deficiencies associated with the prior art.

Applicant has surprisingly found that maintaining contact of EPL cells with a conditioned medium as hereinafter described, preferably in cell aggregates grown in suspension, may be used to produce an at least partially differentiated cell type equivalent to embryonic neurectoderm, which can differentiate further to all neural cell types.

In a first aspect of the present invention there is provided a method of producing neurectoderm cells, which method includes

providing

a source of early primitive ectoderm-like (EPL) cells;

a conditioned medium as hereinafter described; or an extract therefrom exhibiting neural inducing properties; and

contacting the EPL cells with the conditioned medium or extract, for a time sufficient to generate controlled differentiation to neurectoderm cells.

The neurectoderm cells so formed may be characterised in that they are synchronous, homogeneous and their formation parallels the formation of neurectoderm in vivo. The neurectoderm is formed in response to molecules of biological origin and has apparently unrestricted neurectoderm differentiation capability.

As used herein, the term “neurectoderm” refers to undifferentiated neural progenitor cells substantially equivalent to cell populations comprising the neural plate and/or neural tube. Neurectoderm cells referred to herein retain the capacity to differentiate into all neural lineages, including neurons and glia of the central nervous system, and neural crest cells able to form all cell types of the peripheral nervous system.

The neurectoderm cells so formed may be characterised as “early”, for example the neurectoderm cells exhibit neural plate-like characteristics.

This is indicated by upregulation of expression of Gbx2, an early neurectoderm marker.

In a preferred aspect, the neurectoderm cells may be further cultured in a suitable culture medium while neurectoderm cells are formed that may be characterised as “late”, for example the neurectoderm cells exhibit neural tube-like characteristics.

This is indicated by down regulation of Gbx2.

By the term “suitable culture medium” as used herein we mean a culture medium which is suitable for culturing neurectoderm cells. Desirably the culture medium excludes foetal calf serum (FCS). Desirably the culture medium does not include the conditioned medium as hereinbefore described.

Accordingly in a preferred embodiment of this aspect of the present invention, the method includes further providing a suitable culture medium as hereinbefore defined, and

further culturing the early neurectoderm cells in the presence of the suitable culture medium while late neurectoderm cells are formed.

Preferably the late neurectoderm cells so produced exhibit neural tube-like characteristics.

In one form, the method may include the preliminary steps of

providing

a source of pluripotent cells,

a source of a biologically active factor including

a low molecular weight component selected from the group consisting of proline and peptides including proline and functionally active fragments and analogues thereof; and

a large molecular weight component selected from the group consisting of extracellular matrix proteins and functionally active fragments or analogues thereof, or the low or large molecular weight component thereof; and

contacting the pluripotent cells with the biologically active factor, or the large or low molecular weight component thereof, or the conditioned medium, or the extracellular matrix and/or the low molecular weight component, to produce early-primitive ectoderm-like (EPL) cells.

The source of the biologically active factor includes a partially or substantially purified form of the biologically active factor, a conditioned medium including the low and/or large molecular weight component thereof; or an extracellular matrix including a large molecular weight component thereof and/or the low molecular weight component, as described in WO99/53021 above.

The pluripotent cells from which the EPL cells may be derived, may be selected from one or more of the group consisting of embryonic stem (ES) cells, in vivo or in vitro derived ICM/epiblast, in vivo or in vitro derived primitive ectoderm, primordial germ cells (EG cells), teratocarcinoma cells (EC cells), and pluripotent cells derived by dedifferentiation or by nuclear transfer. EPL cells may also be derived from differentiated cells by dedifferentiation.

The step of contacting the pluripotent cells with the biologically active factor, etc. to produce EPL cells may be conducted in any suitable manner. For example, EPL cells may be generated in adherent culture or as cell aggregates in suspension culture. It is particularly preferred that the EPL cells are produced in suspension culture in a culture medium such as Dulbecco's Modified Eagles Medium (DMEM), supplemented with the biologically active factor etc. It is also preferred that there is little or no disruption of cell to cell contact (i.e. trypsinisation).

The conditioned medium utilised in the method according to the present invention is described in International patent application WO99/53021, the entire disclosure of which is incorporated herein by reference.

The term “conditioned medium” includes within its scope a fraction thereof including medium components below approximately 5 kDa, and/or a fraction thereof including medium components above approximately 10 kDa

Preferably the conditioned medium is prepared using a hepatic or hepatoma cell or cell line, more preferably a human hepatocellular carcinoma cell line such as Hep G2 cells (ATCC HB-8065) or Hepa-1c1c-7 cells (ATCC CRL-2026), primary embryonic mouse liver cells, primary adult mouse liver cells, or primary chicken liver cells, or an extraembryonic endodermal cell or cell line such as the cell lines END-2 and PYS-2. However, the conditioned factor may be prepared from a medium conditioned by liver or other cells from any appropriate species, preferably mammalian or avian. The conditioned medium MEDII is particularly preferred.

As stated above, a neural inducing extract from the conditioned medium may be used in place of the conditioned medium. Optionally, the neural inducing extract does not include the biologically active factor conditioned medium or the large or low molecular weight component thereof. The term “neural inducing extract” as used herein includes within its scope a natural or synthetic molecule or molecules which exhibit(s) similar biological activity, e.g. a molecule or molecules which compete with molecules within the conditioned medium that bind to a receptor on EPL cells responsible for neural induction.

In a preferred aspect of the present invention, in the neurectoderm cell production method, the further culturing step is conducted in the presence of a growth factor from the FGF family.

The growth factor from the FGF family, when present, may be of any suitable type. Examples include FGF2 and FGF4.

Accordingly, it should be understood that the term “EPL cells” refers to cells derived from pluripotent cells that retain pluripotency and are converted to and/or maintained as cells that express Oct4 and Fgf5 by:

(a) the biologically active factor as hereinbefore described or the large or low molecular weight component thereof;

(b) a conditioned medium as hereinbefore described; or

(c) An extracellular matrix as hereinbefore described in the presence or absence of the low molecular weight component.

The step of contacting the EPL cells with the conditioned medium may be conducted in any suitable manner. For example neurectoderm cells may be generated in adherent culture or as cell aggregates in suspension culture. Preferably the neurectoderm cells are produced in suspension culture in a culture medium such as DMEM, supplemented with the conditioned medium or-extract. Preferably the cells are cultured for approximately 1 to 7 days, more preferably approximately 3 to 7 days, most preferably approximately 5 days.

Again, a conditioned medium as hereinbefore described may be used to derive and maintain the neurectoderm cells or the conditioned medium may be fractionated to yield an extract therefrom exhibiting neural inducing properties, which may be added alone or in combination to other media to provide the neurectoderm cell deriving medium. The conditioned medium may be used undiluted or diluted (e.g. approx. 10-80%, preferably approximately 40-60%, more preferably approximately 50%).

After day 3, the conditioned medium is preferably replaced with a suitable culture medium as hereinbefore described.

It is optional that the suitable culture medium also includes a growth factor from the FGF family. The concentration of the growth factor from the FGF family is preferably in the range approximately 1 to 100 ng/ml, more preferably approximately 5 to 50 ng/ml. When FGF4 or FGF2 is used, its concentration is preferably approximately 20 ng/ml.

In a further preferred form of this aspect of the invention, the method includes the further step of

identifying the neurectoderm cells by procedures including gene expression markers, morphology and differentiation potential.

The conversion of EPL cells to neurectoderm cells is characterised by

down regulation of expression of Oct4 relative to embryonic stem (ES) cells; and absence of expression of brachyury, and one or more of

up regulation of expression of N-Cam and nestin;

up regulation of expression of Sox1 and Sox2, and

initial up regulation of expression of Gbx2, followed by down regulation thereof as neurectoderm cells persist.

In particular the upregulation of expression of Gbx2 is indicative of neurectoderm cells having neural plate-like characteristics.

The subsequent downregulation of Gbx2 is indicative of cells having neural tube-like characteristics.

Preferably the neurectoderm cell exhibits three of the above characteristics, more preferably four.

For example, the following gene expression profile is evident in neurectoderm cells according to the present invention.

Down regulation of Oct4 expression (pluripotent cell marker).

Expression of N-Cam (expressed by all neural lineages), nestin (an intermediate filament protein expressed by undifferentiated neural stem cells), Sox1 and Sox2 (expressed in neurectoderm and all undifferentiated neural cells), Gbx2 (expressed in neural plate and open neural tube).

Expression of the neural genes Otx1, Mash1, En1 and En2. They may also express Pax3 and Pax6.

Marker genes which may be used to assess the conversion of pluripotent cells to neurectoderm cells and neural lineages include known markers such as Gbx2, Sox1, Sox2, nestin, N-Cam, Oct4 and brachyury. Markers down regulated during the transition from pluripotent cells to neurectoderm include Oct4. Markers up regulated during this transition include Gbx2, Sox1, Sox2, nestin and N-Cam. Markers not expressed during this transition include brachyury (a marker of mesoderm). As neurectoderm cells persist in culture Gbx2 may be down regulated.

The neurectoderm cells according to the present invention may also express the neural genes Otx1, Mash1, En1 and En2. They may also express Pax3 and Pax6.

As stated above, in a further aspect of the present invention there is provided a neurectoderm cell derived in vitro.

The neurectoderm cell may exhibit two of more of the following characteristics

down regulation of Oct4 expression;

expression of N-CAM and nestin;

expression of Sox1 and Sox2

expression of Gbx2;

expression of neural genes, as described above.

In particular the neurectoderm cell exhibits initial upregulation of Gbx2 indicative of early neurectoderm cells having neural plate-like characteristics.

The neurectoderm cell exhibits subsequent down regulation of Gbx2 indicative of late neurectoderm cells having neural tube-like characteristics,

the late neurectoderm being further characterised by the substantial absence of patterning marker expression;

and up regulation of neural genes

The neurectoderm cell according to the present invention has the capacity to differentiate into all neural lineages.

As discussed below, the neurectoderm cell according to the present invention may disperse and differentiate in vivo following brain implantation. In particular following intraventricular implantation, the cell is capable of dispersing widely along the ventricle walls and moving to the sub-ependymal layer. The cell is further able to move into deeper regions of the brain, including into the uninjected side of the brain into sites that include the thalamus, frontal cortex, caudate putamen and colliculus.

During embryogenesis in vivo, neurectoderm cells respond to positional signals that lead to the formation of specific differentiated neural cell types. As part of the response to positional signals, position-dependent gene expression is initiated in neurectoderm cells in restricted locations along the rostro-caudal and dorso-ventral axes within the developing nervous system. These genes serve as markers of positional responses, indicative of future developmental restriction.

Surprisingly, neurectoderm cells according to the present invention do not express the patterning markers HoxB1, Hoxa7, Krox20, Nkx2.2 and Shh. This may distinguish neurectoderm cells produced according to the present invention from neurectoderm cells produced from other sources including for example foetal and adult primary tissues including neural stem cells. Accordingly, the potential for neural development of these cells is expected to be unrestricted, and thus retain the ability to differentiate into all neural cell types, including neuronal cells, glial cells and neural crest cells.

During embryonic development in vivo, HoxB1 is expressed with Phombomere 4, Hoxa7 is expressed in the posterior ectoderm and trunk, Krox20 is expressed in early neural plate, and a more posterior domain, these domains coinciding with later position of rhombomeres 3 and 5, Nkx 2.2 expressed in the developing forebrain and Shh expressed initially in the ventral midbrain, and later extending in a strip of ventral tissue from the rostral limit of the forebrain to the caudal regions of the spine.

If has further been found that by growing EPL cells in suspension culture in presence of factors in the conditioned medium MEDII for a time insufficient to form neurectoderm cells, cells exhibiting characteristics of definitive ectoderm may be produced. Definitive ectoderm cells express lower levels of Oct4 than pluripotent cells, and do not express the neural lineage marker Sox1.

Neurectoderm formation in vivo proceeds via the formation of definitive ectoderm. Although no markers exist for definitive ectoderm in vivo, gene expression analysis identified a population of cells in EPL cells programmed to form neurectoderm which expressed low levels of Oct4 and failed to express neurectodermal markers. See International patent application “Ectodermal Cell Production”, to Applicants, filed on even date. These cells were present transiently between primitive ectoderm (high Oct4 expression, low Sox1, Gbx2 expression) and neurectoderm (high Sox1, Gbx2 expression, low Oct4 expression) and suggest that neurectoderm formation proceeds via an intermediate population which may represent definitive ectoderm.

The definitive ectoderm may exhibit substantially no Sox1 expression.

In a further aspect of the present invention there is provided a method for maintaining neurectoderm cells in vitro in cell populations that are predominantly homogeneous, which method includes

providing

neurectoderm cells produced as described above; and

a suitable culture medium as hereinbefore described;

further culturing the neurectoderm cells in the culture medium to form aggregates of neurectoderm cells.

Preferably the further culturing step begins at day 3 or later.

In a further aspect of the invention there is provided methods for producing differentiated or partially differentiated cells from neurectoderm cells, which method includes

providing

neurectoderm cells as hereinbefore described, and

a suitable culture medium;

further culturing the cells in the presence or absence of a growth factor from the FGF family, and optionally in the presence of additional growth factors and/or differentiation agents, to produce the differentiated or partially differentiated cells.

Preferably the cells produced are cells selected from the group consisting of neuronal cell precursors, neural crest cells, glial cell precursors, or differentiated neurons or glial cells.

The neurectoderm cells may differentiate to form neuronal cells with high frequency.

The step of further culturing the neurectoderm cells in the presence or absence of a growth factor from the FGF family and in the presence of additional growth factors and/or differentiation agents, may be conducted in any suitable manner. Preferably the cellular aggregates or explants cultured in the presence or absence of a growth factor from the FGF family in the presence of additional growth factors and/or differentiation agents. For example, differentiated or partially differentiated cells may be generated in adherent culture or as cell aggregates in suspension culture. Preferably the cells are cultured for approximately a further 3 hours to 10 days, more preferably approximately 1 to 6 days.

The concentration of the growth factor from the FGF family if included is preferably in the range approximately 1 to 100 ng/ml, more preferably approximately 5 to 50 ng/ml. When FGF4 or FGF2 is used, its concentration is preferably approximately 10 ng/ml.

In a preferred embodiment the neurectoderm cells may differentiate in a controlled manner to form predominantly homogeneous populations of neural crest cells.

The additional neurectoderm cell culture step is accordingly preferably conducted in the presence of a Protein Kinase Inhibitor, for example staurosporine.

Staurosporine is a Protein Kinase C inhibitor known to induce neural crest formation from premigratory neural cells in quail (Newgreen and Minichiello, 1996).

The conversion of neurectoderm cells to neural crest cells is characterised by a change in morphology, cell migration and up regulation of expression of Sox10.

A suitable culture medium may include Ham's F12 nurient mixture containing, e.g. 3% FCS and 10 nm/ml FGF2. Plating may occur onto plasticware coated with cellular fibronectin (e.g. 1 μg/cm²).

Culture medium may be supplemented with for example 1 μM to 200 μM staurosporine, preferably 25 μM staurosporine in a suitable solvent (eg DMSO).

In an alternative embodiment, the neurectoderm cells may differentiate in a controlled manner to form predominantly homogeneous populations of glial cells.

The differentiation of neurectoderm to glial cells is accordingly preferably conducted

in a first stage, in the presence of laminin, an FGF growth factor and an EGF growth factor; and

in a second stage, in the presence of a PDGF growth factor and in the substantial absence of EGF, FGF and laminin.

For example neurectoderm aggregates or explants are preferably grown in adherent culture in medium that includes a member of the FGF family (eg; FGF2 or FGF4 10 ng/ml) and EGF (eg; 20 ng/ml) and laminin (eg; 1 to 3 μg/ml) for ˜3 d then cultured for further 2 to 3 d in the absence of EGF, FGF and laminin and presence of PDGF (eg PDGF-M, 10 ng/ml).

The conversion of neurectoderm cell to glial cells is characterised by a change in morphology and up regulation of expresion of the cell surface marker GFAP.

Accordingly, in a further aspect of the present invention, there is provided a partially differentiated neuronal cell, or a terminally differentiated neuronal cell, a partially differentiated neural crest cell, or a terminally differentiated neural crest cell, a partially differentiated glial cell, or a terminally differentiated glial cell, produced by the method described above or derived from neurectoderm cells as hereinbefore described.

Preferably the cells are present as a predominantly homogeneous population.

In a preferred aspect there is now provided

a substantially homogeneous neural crest cell population obtained in vitro exhibiting two or more of the following characteristics:

neural crest cell morphology;

cell migration; and

expression of Sox10.

In a further preferred aspect there is provided

a substantially homogeneous glial cell population obtained in vitro exhibiting one or both of the following characteristics:

glial cell morphology; and

expression of the cell surface marker GFAP.

Preferably the substantially homogeneous glial cell population includes glial cell progenitors and terminally differentiated glial cells.

In a further aspect of the present invention, there is provided a method of producing genetically modified neurectoderm cells or their partially or terminally differentiated progeny, said method including

providing

pluripotent cells,

a source of early primitive ectoderm-like (EPL) cells; and

a conditioned medium as hereinbefore defined, or an extract therefrom exhibiting neural inducing properties

modifying one or more genes in the EPL cells; and

contacting the genetically modified EPL cells with the conditioned medium or extract to produce genetically modified early neurectoderm cells.

Preferably, the method further includes providing a suitable culture medium as hereinbefore defined, and

further culturing the early neurectoderm cells in the presence of the suitable culture medium for a time sufficient to form late neurectoderm cells.

More preferably, the further culturing step is conducted in the presence of a growth factor from the FGF family.

Alternatively, ES cells may be genetically modified before conversion to EPL cells and differentiation to neurectoderm, or neurectoderm cells or their partially or terminally differentiated progeny may be produced as hereinbefore described and then genetically modified.

Accordingly, in a still further aspect of the present invention, there is provided a method of producing genetically modified neurectoderm cells, which method includes

providing

a source of genetically modified pluripotent cells;

a source of a biologically active factor including

a low molecular weight component selected from the group consisting of proline and peptides including proline and functionally active fragments and analogues thereof; and

a large molecular weight component selected from the group consisting of extracellular matrix portions and functionally active fragments or analogues thereof, or the low or large molecular weight component thereof;

a conditioned medium as hereinbefore defined; or an extract therefrom exhibiting neural inducing properties;

contacting the pluripotent cells with the source of the biologically active factor, or the large or low molecular weight component thereof, to produce genetically modified early primitive ectoderm-like (EPL) cells; and

contacting the genetically modified EPL cells with the conditioned medium or extract to produce genetically modified early neurectoderm cells.

The method preferably further includes providing a suitable culture medium as hereinbefore defined, and

further culturing the early neurectoderm cells in the presence of the suitable culture medium for a time sufficient to form genetically modified late neurectoderm cells.

More preferably, the further culturing step is conducted in the presence of a growth factor from the FGF family.

Modification of the genes of these cells may be conducted by any means known to the skilled person which includes introducing extraneous DNA, removing DNA or causing mutations within the DNA of these cells. Modification of the genes includes any changes to the genetic make-up of the cell thereby resulting in a cell genetically different to the original cell.

The genetically modified or unmodified neurectoderm cells of the present invention and the differentiated or partially differentiated cells derived therefrom are well defined, and can be generated in amounts that allow widespread availability for therapeutic and commercial uses. The cells have a number of uses, including the following:

use in human cell therapy to treat and cure neurodegenerative disorders such as Parkinson's disease, Huntington's disease, lysosomal storage diseases, multiple sclerosis, memory and behavioural disorders, Alzheimer's disease and macular degeneration, and other pathological conditions including stroke and spinal chord injury. For example genetically modified or unmodified neurectoderm cells or their differentiated or partially differentiated progeny may be used to replace or assist the normal function of diseased or damaged tissue. For example in Parkinson's disease the dopaminergic cells of the substantia nigra are progressively lost. The dopaminergic cells in Parkinson's patients could be replaced by implantation of neurectodermal neural cells, produced in the manner described in this application.

use of neural crest cells for the derivation of cells for the treatment of spinal cord disorders and Schwann cells for the treatment of multiple sclerosis.

use to produce cells, tissues or components of organs for transplant. For example neural crest cells retain the capacity to form non-neural cells, including cartilage and connective tissue of the head and neck, and are potentially useful in providing tissue for craniofacial reconstruction.

use in human gene therapy to treat neuronal and other diseases. In one approach neurectoderm cells or their differentiated and partially differentiated products may be genetically modified; eg; so that they provide functional biological molecules. The genetically modified cells can be implanted, thus allowing appropriate delivery of therapeutically active molecules.

use as a source of cells for reprogramming. For example karyoplasts from neurectoderm or their differentiated or partially differentiated progeny may be reprogrammed by nuclear transfer. Cytoplasts from neurectodermal cells may also be used as vehicles for reprogramming so that nuclear material derived from other cell types are directed along neural lineages. Alternatively neural stem cells may be reprogrammed in response to environmental and biological signals that they are not normally exposed to. For example the differentiation of murine neural stem cells is redirected to form haematopoietic cells (cells of mesodermal lineage), when injected into the bone marrow (eg; Bjornson et al, 1999). Hence neurectoderm cells described herein are potentially capable of forming differentiated cells of non-neural lineages, including cells of mesodermal lineage, such as haematopoietic cells and muscle. Reprogramming technology using neural cells potentially offers a range of approaches to derive cells for autologous transplant. In one approach karyoplasts from differentiated cells are obtained from the patient, and reprogrammed in neurectoderm cytoplasts to generate autologous neurectoderm. The autologous neurectoderm cells or their differentiated or partially differentiated progeny could then be used in cell therapy to treat neurodegenerative diseases. See Australian provisional patent applications PR1348 and PR2126, the entire disclosures of which are incorporated herein by reference. Alternatively neurectoderm could be further dedifferentiated to a pluripotent state by fusion with pluripotent cytoplasts, and subsequently directed along alternative differentiation pathways to form cells of mesodermal or endodermal lineage.

use in pharmaceutical screening for therapeutic drugs that influence the behaviour of neurectoderm cells and their differentiated or partially differentiated progeny. Neurectoderm cells may be particularly appropriate in evaluating the toxicology and teratogenetic properties of pharmaceutically useful drugs, since many birth defects, including spina bifida are caused by failures in neural tube closure.

Use in the identification and evaluation of biological molecules that direct differentiation of neural cells or neural precursors, including patterning molecules.

Use in identifying genes expressed in neurectoderm cells and partially differentiated or differentiated neural cells.

Accordingly, in a further aspect of the present invention, there is provided a method for the treatment of neuronal and other diseases, as described above, which method includes treating a patient requiring such treatment with genetically modified or unmodified neurectoderm cells as described above, or their partially differentiated or terminally differentiated progeny, through human or animal cell or gene therapy.

In a still further aspect of the present invention, there is provided a method for the preparation of tissue or organs for transplant, which method includes

providing neural crest cells or neurectoderm produced as described above; and

culturing the neural crest cells to produce neural or non-neural cells and the neurectoderm cells to produce neural cells.

The present invention will now be more fully described with reference to the accompanying examples and drawings. It should be understood, however, that the description following is illustrative only and should not be taken in any way as a restriction on the generality of the invention described above.

In the figures:

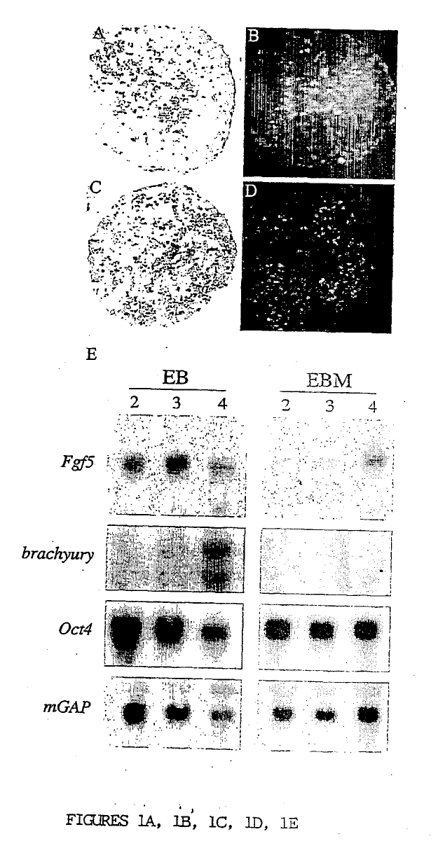

FIG. 1

Analysis of EB⁴ and EBM⁴

A-D. 7 μm sections of paraffin embedded EBM⁴ (A, B) and EB⁴ (C, D) stained with haematoxylin; eosin (A, C) and Hoescht 22358 (B, D). E. 20 μg RNA from EB²⁻⁴ and EBM²⁻⁴ was analysed for the expression of Fgf5, brachyury, Oct4 and mGAP by Northern blot analysis. Fgf5 transcripts were 2.7 and 1.8 kb (Herbert et al., 1990), brachyury 2.1 kb (Lake et al., 2000), Oct4 1.55 kb (Rosner et al., 1990) and mGAP 1.5 kb. F-K In situ hybridisation analysis of EBM⁴ (F, G, H) and EB⁴ (I, J, K) with deoxygenin labelled antisense probes for Oct4 (F, I), Fgf5 (G, J) and brachyury (H, K).

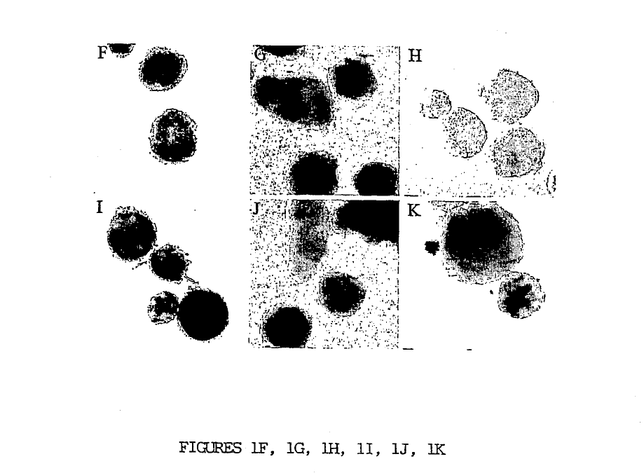

FIG. 2

Differentiation of EBM

Morphology of EBM⁷ (A) and EBM⁹ (B). C. 7 μm section of paraffin embedded EBM⁹ stained with haematoxylin. D. 20 μg RNA from EB⁴⁻⁸ and EBM⁴⁻⁸ was analysed for the expression of Oct4 and mGAP. Oct4 transcripts were 1.55 kb (Rosner et al., 1990) and mGAP 1.5 kb. E. In situ hybridisation analysis of seeded EBM⁷ two days post-seeding with deoxygenin labelled antisense probes for Oct4.

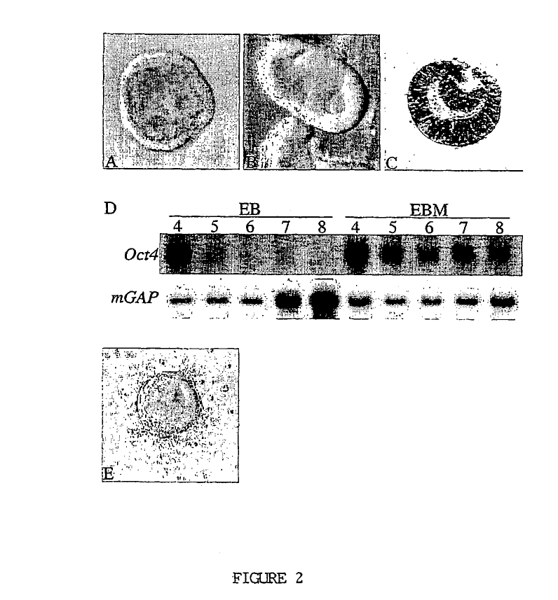

FIG. 3

ES Cells Differentiated as EBM Form Neurectoderm

A, B. In situ hybridisation analysis of seeded EBM⁷ two days post-seeding with deoxygenin labelled antisense probes for Sox1 (A) and Sox2 (B). C, D. Immunohistochemical analysis of seeded EBM⁷ two days post-seeding with antibodies directed against nestin (C) and NCam (D). Nestin immunoreactivity was detected with an enzymatic reaction for the presence of the alkaline phosphatase conjugated secondary antibody. NCam immunoreactivity was detected with a FITC labelled secondary antibody and fluorescent microscopy. E, F. individual EBM were seeded on day 7 of development and assayed on days 8, 10 and 12 for the presence of neurons (E) and beating cardiocytes (F). Neurons and beating cardiocytes were identified morphologically.

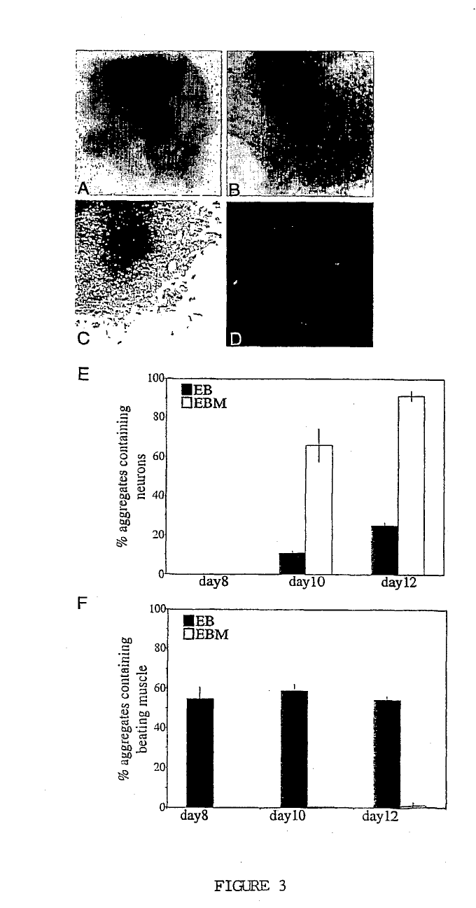

FIG. 4

Neural Formation by Clonal ES Cell Lines

9 clonally derived ES cell lines were differentiated as EB in IC:DMEM and as EBM in IC:DMEM+50% MEDII. Individual cell aggregates were seeded on day 7 of development and scored for the presence of neurons on day 14 and the percentage of aggregates forming neurons was averaged. Neurons were identified morphologically. For each clonal variant and condition n>48.

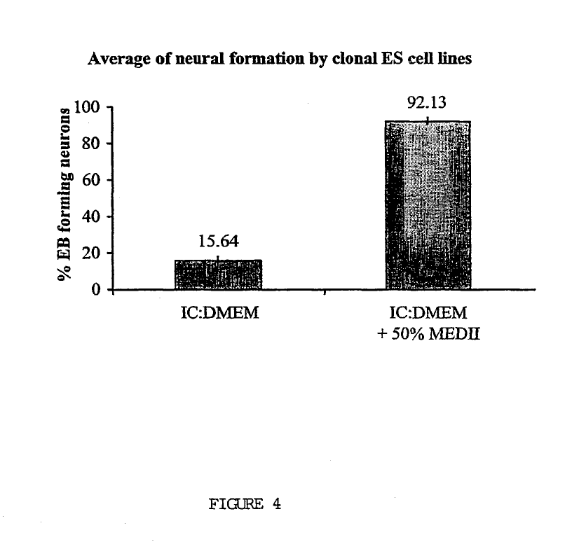

FIG. 5

EDII Induces Neuron Formation from EPL Cells

A. EPL cells were differentiated as cellular aggregates in IC:DMEM (EPLEB) or IC:DMEM+50% MEDII (EPLEBM). On day 7 individual aggregates were seeded and assessed for the formation of beating cardiocytes (n=48). The experiment was repeated three times. B. ES and EPL cells were differentiated as cellular aggregates in IC:DMEM (EB, EPLEB) or IC:DMEM+50% MEDII (EBM, EPLEBM). On day 7 individual aggregates were seeded and assessed for the formation of neurons (n=48). The experiment was repeated three times.

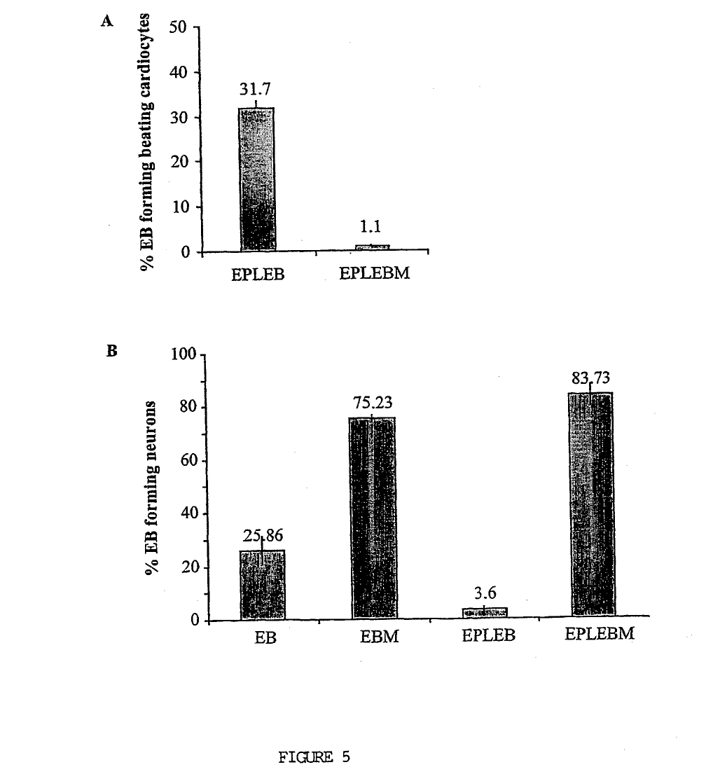

FIG. 6

EBM comprise a Homogeneous Population of Neurectoderm

A, B. EBM⁹ were analysed by wholemount in situ hybridisation for the expression of Sox1 (A) and Sox2 (B). Stained EBM9 were embedded in paraffin wax and 7 μm sections analysed for homogeneity of gene expression. C. Flow cytometry of dissociated EBM¹⁰ and EB¹⁰ analysed for expression of the cell surface antigen NCam. NCam positive cells were identified by comparison with cell populations that had been stained with secondary antibody only (data not shown). Cells staining with intensities greater than the secondary antibody only control were determined to be expressing NCam, and are indicated by the bar.

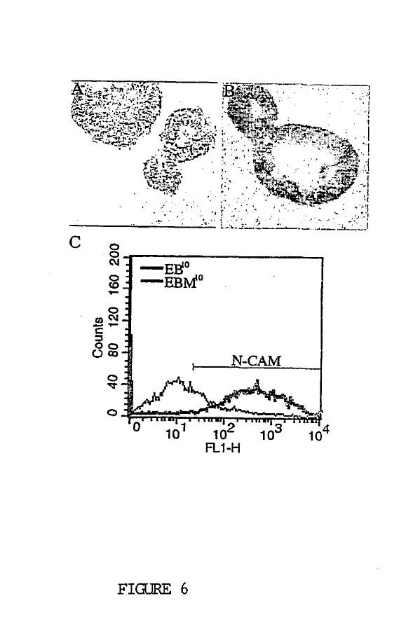

FIG. 7

Gbx2 is Temporally Regulated During EBM Differentiation

In situ hybridisation analysis of seeded EBM⁷ 1 (A, D), 2 (B, E) and 3 (C, F) days post-seeding with deoxygenin labelled antisense probes for Sox1 (A, B, C) and Gbx2 (D, E, F).

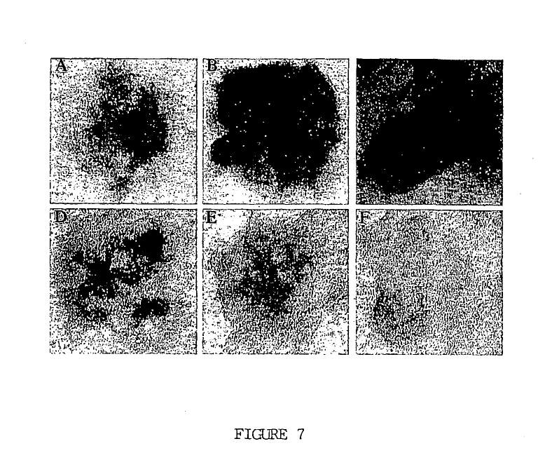

FIG. 8

Expression of Positionally Specified Genes in EBM9

cDNA was synthesised from 1 μg of total RNA isolated from EB⁹ (EB), EPLEB⁹ (EPLEB), EBM⁹ (EBM) and a day 10 mouse embryo (day 10) and used as a template for PCR analysis of the genes denoted. Expression of Actin was used as a positive control. Primer sequences and product sizes can be found in example 3.

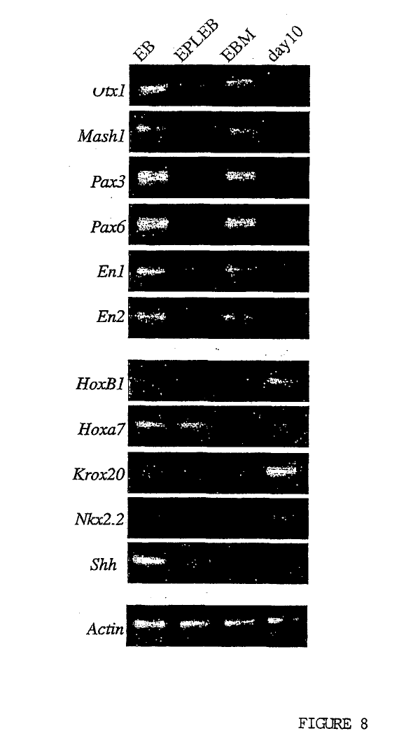

FIG. 9

ES Cell-Derived Neurectoderm can be Directed Down Neural Crest and Glial Lineages

A-D. EBM⁹ explants were seeded onto cellular fibronectin treated tissue culture plastioware in medium supplemented with 25 nM staurosporine/0.1% DMSO (A, C, D) or 0.1% DMSO alone (B). Cultures were examined after 3 (A, B) or 48 hours (C, D). In situ hybridisation analysis of EBM⁹ explants with deoxygenin labelled antisense probes for Sox10 (D). E-H. EBM⁹ explants were seeded onto poly-L-omithine treated tissue culture plasticware in medium supplemented with 10 ng/ml FGF2, 20 ng/ml EGF and 1 μg/ml laminin (E, F) followed by culture in medium supplemented with 10 ng/ml PDGF-M (G, H). Cultures were examined after 2 (A) or 4 (F), and 6 (G, H) days. H. Immunohistochemistry of EBM⁹ explants with antibodies directed against glial fibrillary acidic protein (GFAP). GFAP immunoreactivity was detected with an enzymatic reaction for the presence of the alkaline phosphatase conjugated secondary antibody

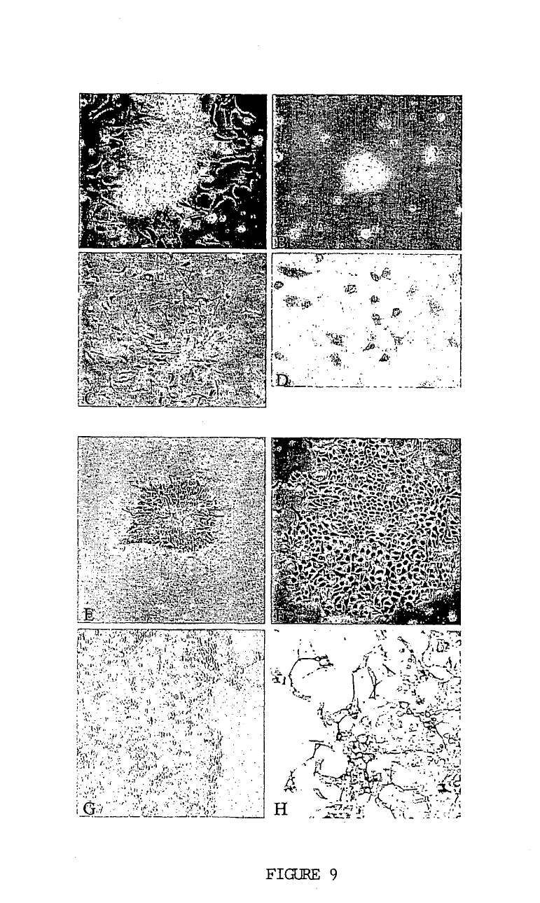

FIG. 10

GFP Positive Cells After 2 Weeks in Rat Brain

Light (left) and fluorescent (right) microscope (Nikon TE300) images of GFP positive cells (arrow, right) in the ependyma of the left lateral ventricle of the rat brain implanted with 200,000 EBM7 at 2 weeks. The brain section was 0.5 mm thick and the picture was taken at 40 times magnification.

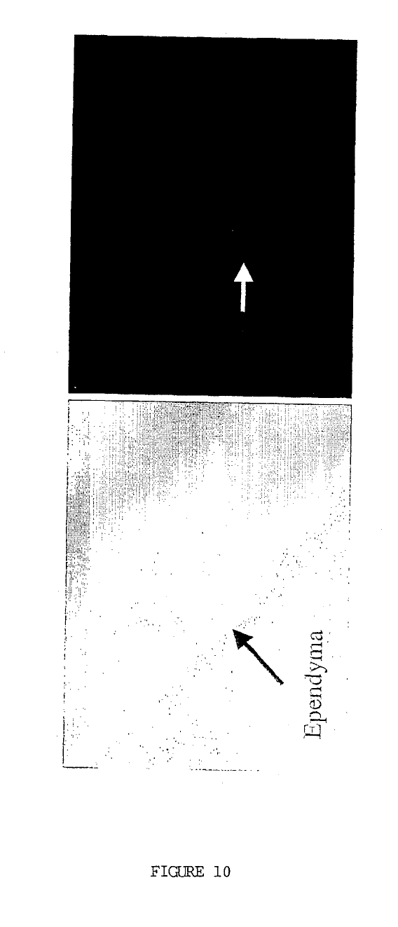

FIG. 11

GFP Positive Cells After 4 Weeks in Rat Brain

Light (left) and fluorescent (right) microscope (Nikon TE300) images of dispersed GFP positive cells (arrow, right) in the sub ependymal layer of the left lateral ventricle of the rat brain injected with 200,000 EBM7 at 4 weeks. The brain section was 0.5 mm thick and the picture was taken at 40 times magnification.

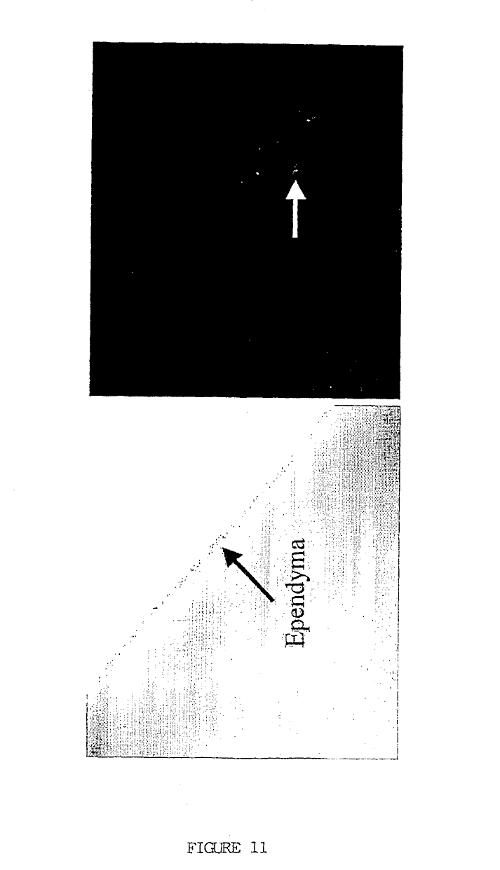

FIG. 12

GFP Positive Cells at 8 Weeks in Rat Brain

Confocal images of GFP positive cells located in the caudate putamen of the rat brain injected with EBM⁷ at 8 weeks. Neural processes (arrows) indicate cell differentiation. The brain section was 0.5 mm thick and the field of view is 216×144 μm.

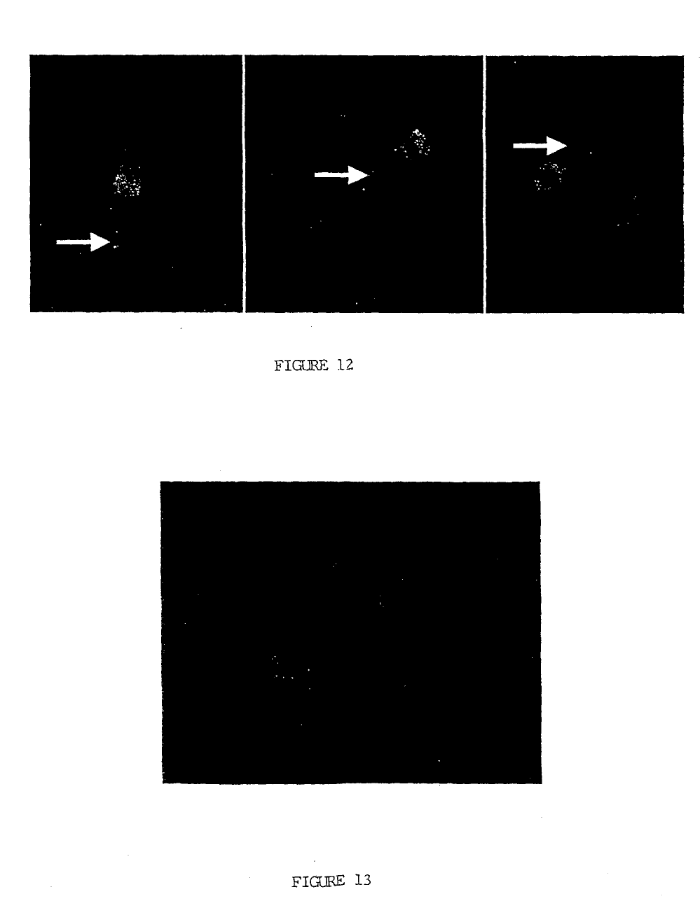

FIG. 13

GFP Positive Cells at 16 Weeks in Rat Brain

Confocal image of GFP positive cells located in the thalamus of the rat brain injected with EBM⁷ at 16 weeks. The brain section was 0.5 mm thick and the field of view is 216×144 μm.

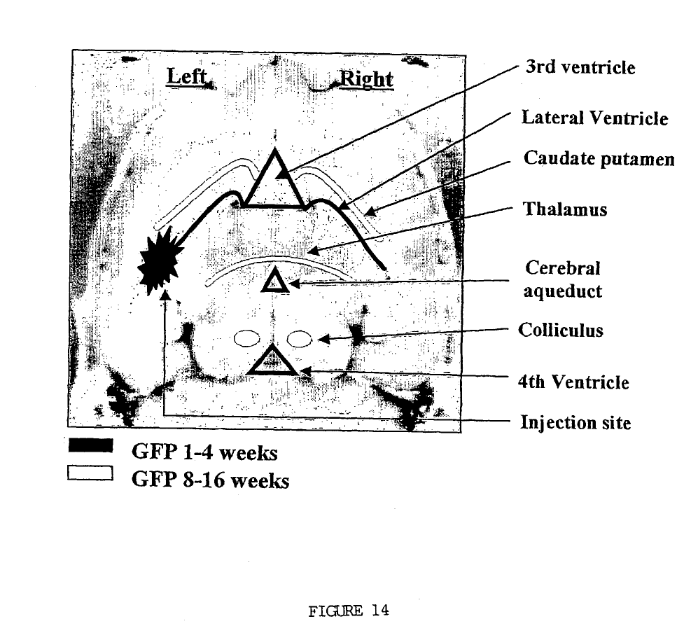

FIG. 14

The Distribution of GFP Positive Cells (EBM⁷ and EBM¹⁰) in the Rat Brain Over Time

The black represents areas where cells were identified up to 4 weeks post injection and the white represents regions of the brain where the cells were located at times up to 16 weeks post injection. Note that, the two dimensional slice through the rat brain does not depict all the labeled regions.

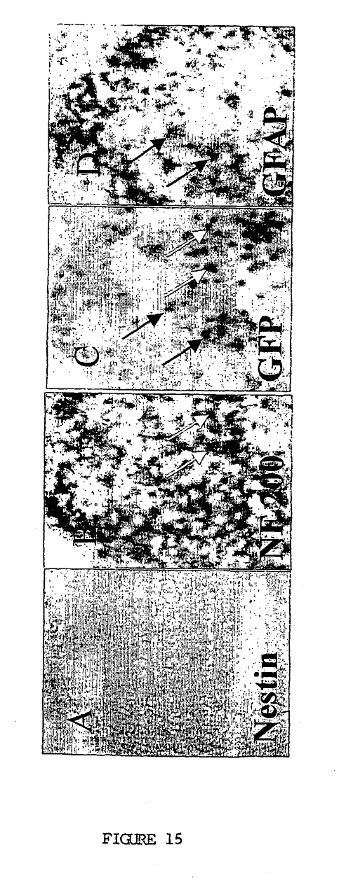

FIG. 15

Cell Differentiation at 2 Weeks in Rat Brain

Immunohistochemical analysis of cells in serial sections (7 μm) of rat caudate putamen 14 days after EBM⁷ implantation. Sections were stained for the expression of nestin (A), NF200 (B), GFP (C) and GFAP (D). The arrows represent regions of GFP+NF200 co-expression (white arrows) and GFP+GFAP co-expression (black arrow). Pictures were taken at 400 times magnification.

### EXAMPLE 1

### Formation of Neurectoderm Cells by Directed Differentiation of Pluripotent Cells in vitro

Methods

Cell culture

ES cell lines E14 (Hooper et al., 1987) and D3 (Doetschman et al., 1985) were used in this study. Routine culture of ES and EPL cells and production of MEDII and sfMEDII conditioned medium were as described in Rathjen et al. (1999).

Formation of GFP Expressing D3 ES Cell Lines

D3 ES cells expressing enhanced green fluorescent protein (EGFP; Clontech) under the control of the constitutive EF1α promoter were formed by transfection with pFIRES+EGFP (obtained from Dr. S. Dalton, Department of Molecular Biosciences, Adelaide University, Australia). 5×10⁶ D3 ES cells in 0.5 ml HBS+glucose (20 mM HEPES, 140 mM NaCl, 5 mM KCl, 7 mM Na₂HPO₄, 6 mM glucose, 0.1 mM β-mercaptoethanol (β-ME)) were transfected by electroporation with 15 μg plasmid DNA (960 μFd; 210 V) using a BioRad Gene Pulser. Following transfection, cells were allowed to recover at room temperature for 10 minutes before plating and culturing at a density of 4.4×10⁴ cell/cm² in IC DMEM+LIF (Dulbecco's Modified Eagles Medium (DMEM; Gibco BRL #12800) supplemented with 10% foetal calf serum (FCS; Commonwealth Serum Laboratories), 40 mg/ml gentamycin, 1 mM L-glutamine, 0.1 mM β-ME and 1000 units of LIF). After 24 hours the medium was changed to IC DMEM+LIF supplemented with 1.2 μg/ml puromycin (Sigma). Stable transformants were picked after 8 days of selection. Single colonies were picked, expanded and assessed morphologically for the expression of EGFP in pluripotent cells and differentiated derivatives. EGFP fluorescence was detected on a Nikon TE300 inverted microscope using a FITC filter. PS Formation of Cell Aggregates

All cell aggregates were formed from single cell suspensions (1×10⁵ cells/ml) of ES or EPL cells cultured in bacterial petri dishes. ES cell and EPL cell embryoid bodies (EB and EPLEB respectively) were formed as described in Lake et al. (2000). EBM, cell aggregates formed and maintained in MEDII, were formed from ES cells aggregated in IC:DMEM (DMEM (Gibco BRL #12800) with 10% foetal calf serum (FCS; Commonwealth Serum Laboratories), 40 mg/ml gentamycin, 1 mM L-glutamine and 0.1 mM B-mercaptoethanol (B-ME)) supplemented with 50% MEDII. Aggregates were divided 1 in 2 on days 2 and 4, and medium was changed on days 2 and 4 and then daily until collection. In early experiments 10-20 ng/ml FGF4 was added to the medium from day 4, however this did not influence the outcome of differentiation and was omitted in later experiments. The time in days from formation of aggregates was denoted by superscript with the day of formation denoted as day 0. For example, EBM 5 days after formation are represented as EBM⁵.

For continued suspension culture of EB and EBM, aggregates on day 7 were transferred to serum free medium (50% DMEM, 50% Hams F12 (Gibco BRL # 11765) supplemented with 1× ITSS supplement (Boehringer Mannhiem) and 10 ng/ml FGF2 (Peprotech Inc.)).

For adherent culture, aggregates were seeded onto gelatin treated tissue culture grade plasticware (Falcon) on day 7 of development in 500 μl DMEM supplemented with 10% FCS (Commonwealth serum Laboratories). On day 8 medium was removed and replaced with 50% DMEM, 50% Hams F12 supplemented with 1× ITSS (Boehringer Mannhiem).

Analysis of Differentiation Potential of Cells Within Cellular Aggregates

EB⁷ and EBM⁷ were seeded as described above and assessed on days 8, 10, 12 and 14 for the presence of neurons, identified morphologically by the presence of axonal projections (and confirmed by the expression of NF200; data not shown), and beating cardiocytes, identified morphologically by rhythmical contraction of cells within the aggregate.

Gene Expression Analysis

Cytoplasmic RNA was isolated from cellular aggregates using the following method. Cellular aggregates were resuspended in 1 ml extraction buffer (50 mM NaCl, 50 mM Tris.Cl pH 7.5, 5 mM EDTA pH 8.0 and 0.5% SDS) and acid washed glass beads (40 mesh; BDH) were added to the meniscus. Suspensions were vigorously vortexed before the addition of a further 3 ml of extraction buffer and 200 μg/ml proteinase K (Merck) and incubation at 37° C. for 60 minutes. One tenth volume of 3M sodium acetate was added before the suspension was phenol:chloroform extracted. The aqueous phase was added to an equal volume of iso-propanol, chilled to −80° C. for 30 minutes and the nucleic acids pelleted by centrifugation (Jouan bench centrifuge, 3,000 rpm). The pellet was resuspended in DNase I digestion buffer (50 mM Tris.Cl pH 8.0, 1 mM EDTA pH 8.0, 10 mM MgCl, 0.1 mM DTT) and 20 units of RNase free DNase I (Boehringer Mannhiem) added. After incubation at 37° C. for 60 minutes nucleic acids were phenol:chloroform extracted and ethanol precipitated. Northern blot analysis was performed as described in Thomas et al. (1995). DNA probes were prepared from DNA fragments using a Gigaprime labelling kit (Bresagen). DNA fragments used were as described in Rathjen et al. (1999) and Lake et al. (2000).

Wholemount in situ hybridisation on cell layers was performed using the method of Rosen and Beddington (1993) as described in Rathjen et al., (1999). Antisense and sense probes for the detection of Oct4, Fgf5 and brachyury were synthesised as described in Rathjen et al. (1999). Antisense Sox1 probes were synthesised by T3 RNA polymerase as run-off transcripts from plasmid #1022 linearised with BamHI. Sox1 sense transcripts, used as controls, were obtained from the same plasmid linearised with HindIII and transcribed by T7 RNA polymerase. Sox2 transcripts were generated from a 748 bp Accl/Xbal cDNA fragment cloned into pBluescript SK (obtained from Dr. R. Lovell-Badge, Division of Developmental Genetics, National Institute for Medical Research, Mill Hill, London). Transcripts were generated from AccI and XbaI linearised plasmid transcribed with T3 (anti-sense) and 17 (sense) RNA polymerases respectively.

Histological Analysis

EB⁴ and EBM⁴ were fixed with 4% PFA for 30 minutes before embedding in paraffin wax and sectioning as described in Hogan et al., 1994. 7 μm sections were stained with haematoxylin:eosin as described by Kaufman, 1992 or with Hoescht 22358 (5 μg/ml) in PBS; Sigma) for 5 minutes.

Immunohistochemical Analysis

Cellular aggregates were fixed in 4% paraformaldehyde in PBS for 30 minutes, and dehydrated in sequential 30 minute washes in 50% ethanol and 70% ethanol. Cells were rehydrated to PBS and permeabilised with RIPA buffer (150 mM NaCl; 1% NP-40; 0.5% NaDOC; 0.1% SDS) for 30 minutes, washed in PBS and blocked in the appropriate blocking buffer as described below for 30 minutes. Primary antibodies, diluted in the appropriate blocking buffer, were added and incubated overnight at 4° C. After washing in PBS, aggregates were incubated with alkaline phosphatase conjugated, species specific secondary antibodies directed against the primary antibodies in 100 mM Tris.Cl (pH 7.5), 100 mM NaCl, 0.5% blocking reagent (Boehringer Mannheim). For alkaline phosphatase conjugated secondary antibodies, cellular aggregates were washed in Buffer 2 (100 mM Tris.Cl (pH 9.5), 100 mM NaCl, 5 mM MgCl₂) and antibody conjugates were detected enzymatically with NBT and BCIP (both Boehringer Mannheim) made up in Buffer 2 according to the manufacturers instructions. Aggregates were visualised on a Nikon TE300 using Hoffmann interference contrast optics. For FITC conjugated secondary antibodies, cellular aggregates were washed in PBS and mounted in 80% glycerol containing 5 mg/ml propyl gallate (Sigma). Aggregates were examined on a Nikon TE300 microscope using a FITC filter.

Nestin: Blocking buffer: 10% goat serum, 2% BSA in PBS. Primary antibody: Developmental Studies Hybridoma Bank, reference Rat 401, used at a dilution of 1:100. Secondary antibody: alkaline phosphatase conjugated goat anti-mouse IgG (affinity purified, Rockland) used at a concentration of 1:100.

N-Cam: Blocking buffer 1% FCS, 1 mg/ml BSA, 1% Triton X 100 in PBS. Primary antibody: Santa Cruz Biotech, SC-1507 used at a concentration of 1:20. Secondary antibody: FITC conjugated goat anti-mouse IgM (μ-specific: Sigma) used at a dilution of 1:700.

NF20: Blocking buffer: 10% goat serum, 2% BSA in PBS Primary antibody: anti-neurofilament 200 (Sigma Immunochemicals N-4142) used at a dilution of 1:200. Secondary antibody: alkaline phosphatase conjugated goat anti-rabbit IgG (ZyMax™ grade, Zymed Laboratories Inc.)

Results

Formation of EPL Cells from ES Cells in Suspension

Embryonic stem (ES) cells cultured in the presence of medium conditioned by the human hepatocellular carcinoma cell line HepG2 (MEDII) have been shown to form a second pluripotent cell population, EPL cells (Rathjen et al., 1999). EPL cells demonstrate morphology, gene expression, differentiation potential and cytokine responsiveness distinct from ES cells but characteristic of the post-implantation pluripotent cell population of the mouse embryo, primitive ectoderm (Rathjen et al., 1999.

ES cells can be aggregated in suspension culture. In the absence of exogenous cytokines, such as LIF, aggregated ES cells form structures termed embryoid bodies (EB), which recapitulate many aspects of cell differentiation during early mammalian embryogenesis (Doetschman et al., 1985; Shen and Leder, 1992. Outer cells form extraembryonic endoderm and derivatives while inner cells undergo processes equivalent to formation of the proamnioatic cavity (Coucouvanis and Martin, 1995) and primitive ectoderm (Shen and Leder, 1992), followed by pluripotent cell differentiation into differentiated tissues derived from all three germ layers.

ES cells were aggregated in medium supplemented with 50% MEDII (EBM) and compared to EB development. After 4 days cellular aggregates formed in the presence of MEDII (EBM⁴) could be distinguished from EB⁴ by morphology. Histological analysis of sectioned EB⁴ and EBM⁴ showed EBM⁴ to comprise a multi-cell layer of uniform thickness surrounding a single, internal area of cell death indicated by the presence of pyknotic nuclei (FIGS. 1A, B). No morphologically distinct outer layer of cells reflecting the presence of extraembryonic endoderm could be detected at this or later stages of EBM development. In contrast, EB⁴ were internally disorganised with sporadic, multiple foci of cell death dispersed throughout the aggregates (FIGS. 1C, D). An outer layer of extraembryonic endoderm was apparent at low levels in. EB⁴ and at higher levels in more advanced EB (data not shown).

EB²⁻⁴ and EBM²⁻⁴ were analysed by Northern blot (FIG. 1E) for the expression of Oct4, a marker gene for pluripotent cells (Scholer et al., 1990), and Fgf5, a gene up-regulated in pluripotent cells upon primitive ectoderm formation (Haub and Goldfarb, 1991). Oct4 expression was maintained at high levels throughout these stages of EBM development indicating that pluripotent cell differentiation had not commenced within these aggregates. High level Oct4 expression in EBM⁴ was accompanied by elevated Fgf5 expression, indicating that the pluripotent cells had formed primitive ectoderm. In contrast, highest levels of Oct4 and Fgf5 expression in EB were observed at: days 2-3 and day 3 respectively. Downregulation of both genes in EB⁴ indicated that pluripotent cells within these aggregates had differentiated.

The distribution of pluripotent cells within aggregates was investigated by wholemount in situ hybridisation of EB⁴ and EBM⁴ with Oct4 and Fgf5 antisense probes. Homogeneous expression of Oct4 (FIG. 1F) and Fgf5 (FIG. 1G) within and between individual EBM⁴ aggregates was consistent with the deduced cellular homogeneity of primitive ectoderm within these aggregates and persistence of pluripotent cells to day 4. This contrasted with patchy expression of these markers within and between individual EB⁴ aggregates (FIGS. 1. I, J), consistent with the variable onset and progression of pluripotent cell differentiation within EB described here and by others (Haub and Goldfarb, 1991).

The expression of brachyury, a marker for nascent mesoderm (Herrmann, 1991), was used to confirm the onset of mesodermal differentiation in the aggregates. Brachyury expression was analysed in EBM²⁻⁴ and EB²⁻⁴ by Northern blot (FIG. 1E) and in EBM⁴ and EB⁴ by wholemount in situ hybridisation (FIGS. 1H, K). In EB brachyury expression was upregulated on day 4 of development, coincident with the loss of pluripotence in the aggregates. In contrast brachyury expression could not be detected by either method in EBM²⁻⁴, consistent with the maintenance of Oct4 expression and suggesting a lack of differentiation within these aggregates.

These results suggest that MEDII effected relatively homogeneous and synchronous formation of EPL cells from ES cells in suspension. On day 4 of development EBM comprise a homogeneous population of pluripotent cells that have acquired primitive ectoderm-like gene expression with no detectable associated differentiation.

MEDII has been shown to contain 50-100 units of human LIF (Rathjen et al., 1999). LIF has been shown to retard the developmental progression of EB in vitro (Shen and Leder, 1992). ES cells aggregated and maintained in medium supplemented with 100 units of LIF did not duplicate the morphology or gene expression profile of EBM (data not shown), indicating the importance of additional secreted factors contained within MEDII (Rathjen et al., 1999) for EPL cell induction.

Programmed Formation of Ectodermal and Neurectodermal Lineages by Pluripotent Cell Differentiation in vitro

Continued culture of EBM in medium containing 50% MEDII resulted in the formation of cellular aggregates displaying an unusual and distinct morphology. By day 7>95% of the cellular aggregates within the EBM population comprised a convoluted cell monolayer as shown in FIG. 2A. EBM⁷ transferred to 50% DMEM:50% Hams F12 supplemented with ITSS and 10 ng/ml FGF2 for a further 2 days of culture formed a population in which >95% aggregates comprised a single stratified epithelial sheet (FIG. 2B, C). Cellular aggregates of similar morphology were not detected within the EB⁷ or EB⁹ populations although equivalent cell layers could be detected within a proportion of individual aggregates (data not shown).

Northern blot analysis of EB⁴⁻⁸ and EBM4-8 showed a down-regulation of Oct4 in both populations (FIG. 2D) suggesting differentiation of the pluripotent cells within both populations of aggregates. However, while Oct4 was undetectable in EB after day 5, a low but consistent level of Oct4 expression, 4.2-fold lower than EBM⁴, could be detected in EBM on all days of development after day 5. EBM⁷ were seeded onto gelatin treated tissue culture grade plasticware in DMEM containing 10% FCS and analysed after a further 24 hours culture (EBM) by wholemount in situ hybridisation with an Oct4 anti-sense probe. This analysis failed to detect cells expressing Oct4 at levels equivalent to pluripotent cells (FIG. 2E), which suggested that the Oct4 expression detected by Northern blot analysis represented low level expression by the majority of cells within the population and not expression by a small population of residual pluripotent cells within the aggregates.

As the morphology of EBM⁹ was clearly reminiscent of neurectoderm (FIG. 2C), the expression of a number of neural markers was analysed. EBM⁷ were seeded onto gelatin treated tissue culture grade plasticware in DMEM containing 10% FCS and the medium was changed to 50% DMEM:50% Hams F12 supplemented with ITSS and 10 ng/ml FGF2 after 16 hours. These aggregates were analysed by in situ hybridisation for the expression of Sox1 and Sox2. Sox1 has been shown to delineate the neural plate and is expressed by all undifferentiated neural cells, while Sox2 shows a similar expression pattern but is expressed earlier in embryogenesis (Pevney et al., 1998). Seeded aggregates were also analysed by immunohistochemistry using antibodies directed against nestin, a neurofilament protein expressed in neural progenitor cells (Zimmerman et al., 1994) and N-Cam, a cadherin expressed within the neural system by primitive neurectoderm, neurons and glia (Rutihauser, 1992). Widespread expression of Sox1 and Sox2 was detected within seeded aggregates derived from EBM⁷ (FIGS. 3A, B). In accordance with the acquisition of neural gene expression, both nestin and N-Cam were also expressed widely in these aggregates (FIGS. 3C, 3D).

As a consequence of seeding, cells on the periphery of the seeded aggregates differentiated spontaneously. Individual EBM7 were seeded as described above and assessed on days 8, 10 and 12 for the presence of beating cardiocytes, a differentiated mesoderm derivative, and neurons, a differentiated ectoderm derivative. The differentiation of EBM was compared to EB (FIG. 3E, F). Consistent with the up-regulation of neural specific markers, and lack of brachyury expression, neurons could be seen in the differentiated products of the majority of EBM (91.33%). However, <2% of EBM formed beating cardiocytes. In contrast, EB comprised a mixed population which differentiated into both beating cardiocytes (54.5%) and neurons (24.9%) on day 12 respectively.

Gene expression and differentiation analyses indicate that continued culture of EBM⁴, which comprise an homogeneous population of EPL cells, in the presence of MEDII, programs differentiation of the pluripotent cells to an ectodermal and neurectodermal fate. The great majority of differentiated cells at day 9 expressed neural markers such as Sox1, Sox2, nestin and N-Cam, and spontaneous differentiation resulted in efficient formation of neurons. The absence of brachyury expression and failure of seeded aggregates to form differentiated mesodermal derivatives indicated that elevated ectodermal differentiation was achieved at the expense of mesoderm formation. This directed differentiation to ectoderm/neurectoderm contrasts with the haphazard differentiation observed in EB, and with the mesoderm-specific differentiation reported for EPLEB.

Programming Neural Pluripotent Cell Differentiation with MEDII Overcomes Heterogeneity Associated with EB Differentiation of Different ES Cell Lines

Experimental analysis using EB is restricted by variable differentiation of alternative ES cell lines (our unpublished data). Nine clonal ES cell isolates expressing EGFP were differentiated as EB or EBM. Individual cell aggregates were seeded on day 7 and assessed for the formation of neurons on day 14 (FIG. 4). All clonal lines formed neurons when differentiated as either EB or EBM. When differentiated as EB the formation of neurons varied between 4 and 28% of bodies, with an average of 15.64%+/−2.54. This indicates that the uncontrolled differentiation of pluripotent cells in EB is not consistent between different clonal ES cell isolates. All clonal lines responded to MEDII as previously described by formation of neurectoderm-containing aggregates. Neuron formation was increased to >80% of aggregates, with an average of 92.13%+/−1.944. This result suggests that response to MEDII by differentiation to ectodermal lineages is an inherent property of ES cells and not restricted to a subpopulation within the ES cell population. Further, MEDII-directed differentiation of ES cells as EBM overcame the inherent variability associated with EB differentiation and resulted in uniform, high level production of neurectoderm and neurons.

EPL Cells Differentiate to form Neurons in Response to MEDII

It has been previously reported that EPL cells form neurons poorly, if at all, when differentiated as EB but form elevated levels of nascent and differentiated mesoderm (see International patent application PCT/AU99/00265, above). This has been interpreted as reflecting disrupted signalling from visceral endoderm or visceral endoderm-derived ECM (Lake et al, 2000). EPL cells were formed from ES cells as described, and aggregated and cultured in suspension for 7 days in either IC:DMEM (EPLEB) or IC:DMEM supplemented with 50% MEDII (EPLEBM). On day 7, individual EPL cell embryoid bodies were seeded onto gelatin treated tissue culture plasticware in IC:DMEM. On day 8 the medium was changed to DMEM:F12 and embryoid bodies were cultured for a further 4 days before microscopic inspection for the presence of beating cardiocytes and neurons.

As shown in FIG. 5A, EPL cell embryoid bodies formed beating cardiocytes efficiently (35.25%), consistent with previous reports and gene expression (Lake et al., 2000). In contrast, EPL cell embryoid bodies cultured in the presence of 50% MEDII exhibited drastically lower levels of beating cardiocyte formation (0.9%). Aggregation and differentiation of EPL cells in the presence of MEDII resulted in an up regulation in neuron formation from very low levels in EPLEB (3.6%), to 83.73% in EPLEBM (FIG. 5B). These data suggest that signals contained within MEDII replace those deficient in the EPLEB differentiation environment to direct the pluripotent cells to an ectodermal/neural fate.

Conclusion

These results demonstrate that pluripotent cells can be programmed specifically to an ectodermal and neurectodermal fate by factors within MEDII. The ectodermal cells are formed in the absence of mesodermal cell types, and exhibit a temporal pattern of gene expression equivalent to neurectoderm in vivo. In the embryo these cells are precursors for all neural lineages.

Results of further differentiation in vitro support the conclusion that neurectoderm is formed at high levels and in the absence of mesodermal cells by EBM cultured in the presence of MEDII, since the neurectoderm can give rise to terminally differentiated neural cell types at elevated levels, but not terminally differentiated mesodermal cell types. Aggregates developed from EBM in MEDII are therefore enriched in undifferentiated neural cells.

The formation of neurectoderm described here does not rely on the addition of chemical inducers, such as retinoic acid, or genetic manipulation to promote neural formation. Instead, it relies on biologically derived factors found within the conditioned medium MEDII. Neural progenitors formed in this manner are thought to be differentiated from pluripotent cells in a manner analogous to the formation of neural cells during embryogenesis and are therefore ideal for the production of differentiated neural cells useful for commercial, medical and agricultural applications. Further, in contrast to the formation of limited neural lineages by chemical inducers such as retinoic acid, the identity of neural cell types produced using these methodologies is not likely to be developmentally restricted.

### EXAMPLE 2

EBM Comprise a Homogeneous and Synchronous Population of ES Cell-Derived Neural Progenitors

Methods

Cell Culture and Gene Expression Analysis

EB and EBM were aggregated and cultured as described in example 1. Gene expression analysis was as described in example 1. EBM which had been analysed by whole mount in situ hybridisation staining were prepared for histochemical analysis as follows. Stained EBM were fixed in 4% PFA overnight, washed several times with PBS, 0.1% Tween-20, treated with 100% methanol for 5 minutes and then isopropanol for 10 minutes. Bodies were then treated and embedded as described in Hogan et al. (1994).

DIG labelled Gbx2 riboprobes were generated from pG290 which contains a 290 bp PCR fragment from base 780 to base 1070 of the Gbx2 cDNA (Chapman and Rathjen, 1995) cloned into pGEMT-easy (Promega). Antisense and sense probes were transcribed from Sall or Styl cut pG290 with 17 or T3 RNA polymerase respectively.

Flow Cytometry Analysis

EB¹⁰ and EBM¹⁰ were collected and washed in PBS, then disassociated by incubating for 5 minutes in 0.5 mM EDTA/PBS followed by vigorous pipetting and agitation to a single cell suspension. Cells were washed several times in PBS before fixation with 4% PFA for 30 minutes. Cells were washed with 1% BSA/PBS, resuspended at 1×10⁶ cells/ml, and incubated with antibody directed against N-Cam (Santa Cruz Biotech, SC-1507) at a dilution of 1:2 for 1 hour. Cells were washed with 1% BSA/PBS before incubation with FITC conjugated goat anti-mouse IgM (μ-specific: Sigma) used at a concentration of 1:100. FITC conjugated goat anti-mouse IgM was pre-adsorbed for 1 hour in 1% BSA/PBS before use. Cells were washed in PBS and fixed in 1% PFA for 30 minutes. Data was collected on 1×10⁴ cells on a Benton Dickonson FACScan and analysis performed using CellQuest 3.1.

Results

EBM Comprise a Homogeneous Population of Neural Progenitor Cells

Data presented in example 1 suggested that within the EBM population approaching 100% of the cellular aggregates contained neural progenitor cells. The number of cells within the population expressing neural specific markers was evaluated to assess the homogeneity of differentiation. EBM⁹ were probed by wholemount in situ hybridisation for Sox1 and Sox2 expression and histological sections were examined for homogeneity of expression. Representative sections (FIG. 6A, B) showed that EBM⁹ comprised a morphologically uniform population of cells equivalent to the neurectoderm-like monolayer described in example 1, in which each cell stained positive for expression of Sox1 and Sox2.

To enable comparative quantitation of neurectoderm formation, EBM¹⁰ and EB¹⁰ were disaggregated to a single cell suspension, labelled immunocytochemically with antibodies directed against N-Cam, a cell adhesion molecule expressed strongly in the nervous system (Ronn et al., 1998) and analysed by FACS analysis (FIG. 6C). 95.7% of cells from EBM¹⁰ were scored positive for N-Cam expression, demonstrating relatively uniform differentiation of these aggregates to neural lineages. In comparison, only 42.13% of cells from EB¹⁰ expressed N-Cam, consistent with the established heterogeneity of ES cell differentiation within this system.

Neural Formation Within EBM is Relatively Synchronous and Reflects the Temporal Formation of Neural Lineages in the Embryo

During embryogenesis formation of neurectoderm is characterised by progressive alterations in gene expression. The neural plate, which contains the earliest neural precursors, is characterised by expression of Sox1 within a group of cells on the anterior midline of the embryo (Pevney et al., 1998). This population of cells also expresses the homeobox gene Gbx2 (Wassarman et al., 1997). With continued development the neural plate folds at the midline and the outer edges close to form the neural tube. Sox1 expression is maintained after tube closure but Gbx2 expression is down regulated in the majority of cells of the neural lineage and persists only in a restricted population of cells at the mid-brain/hind-brain boundary (Wassarman et al., 1997).

Wholemount in situ hybridisation of seeded EBM⁸, EBM⁹ and EBM¹⁰ was used to investigate the temporal regulation of Sox1 and Gbx2 in during EBM progression. At day 8, Sox1 was expressed in approximately 50% of the cells within the seeded aggregates (FIG. 7A). The extent of expression was increased in EBM⁹, and evident in the majority of cells within the aggregates on both day 9 and 10 (FIG. 7B, C), indicating homogeneous formation of neurectoderm. Gbx2 was also expressed in approximately 50% of the cells within the seeded aggregates at day 8, but was less abundant in EBM⁹, and was virtually undetectable in EBM¹⁰ (FIGS. 7D, E, F). The loss of Gbx2 expression in aggregates in which Sox1 expression persists recapitulates the temporal regulation of this gene in the developing neural tube of the embryo. Rapid downregulation of Gbx2 expression indicates relative synchrony of the EBM differentiation system, and suggests EBM8 represent cells equivalent to the time of neural tube closure in vivo.

Conclusion

Cellular analysis of gene expression indicates that differentiation of ES cells as EBM results in the formation of a homogeneous population of neural progenitors. Temporal regulation of gene expression indicated relative synchrony of differentiation within and between EBM aggregates, and was conserved in many aspects with formation of the ectodermal/neurectodermal lineages during mammalian embryogenesis. EBM differentiation therefore recapitulates progressive formation of neural plate and neural tube, progenitors for the entire nervous system, from pluripotent cells in the mammalian embryo.

### EXAMPLE 3

### Gene Expression in ES Cell-Derived Neurectoderm Induced by MEDII Indicates that Cells are not Positionally Specified

Methods

Cell Culture and Gene Expression Analysis

EB and EBM were formed and cultured as described in example 1. Gene expression analysis was as described in example 1.

PCR Analysis of Neurectoderm Gene Expression

Total RNA was extracted from cell aggregates as described in Rathjen et al. (2000). cDNA was synthesised from 1 μg of total RNA using Superscript™II First-Strand Synthesis System for RT-PCR (Gibco BRL) following the manufacturers instructions. PCR was performed using Platinum PCR Supermix (Gibco BRL) following the manufacturers instructions. Reactions were performed in a capillary thermocycler (Corbett Research), with cycling parameters as follows; denaturing 94° C., 10 seconds, annealing 55° C., 10 seconds and extension 72° C., 60 seconds. Cycling times were determined for each primer set to be within the exponential phase of amplification. Primers for amplification of actin, En-1, Hoxa7 and Otx1 have been described previously (Okabe et al., 1996). Primer sequences and the length of amplified products were as follows:

En2 (512bp)
(5′ AGGCTCAAGGCTGAG1TVCA 3′:
5′ CAGTCCCC11TGCAGAAAAA 3′),
HoxBl (501 bp)
(5′ CGAAAGGTTGTAGGGCAAGA 3′;
5′ CGGTCTGCTCAGTTCCGTAT 3′),
Shh (502bp)
(5′ GGAACTCACCCCCAATTACA 3′;
5′ GAAGGTGAGGAAGTCGCTGT 3′),
Mash 1 (482bp)
(5′ CGTCCTCTCCGGMCTGAT 3′;
5′ TCCTGCTTCCAAAGTCCATT 3′),
Nkx2.2 (514bp)
(5′ CTCTTCTCCAAAGCGCAGAC 3′;
5′ AACAACCGTGGTAAGGATCG 3′),
Krox2O (502bp)
(5′ GGAGGGCAAAAGGAGATACC 3′;
5′ GGTCCAGTTCAGGCTGAGTC 3′),
Pax3 (502 bp)
(5′ CGTGTCAGATCCCAGTAGCA 3′;
5′ CCTTCCAGGAGGAACTACCC 3′),
Pax6 (500 bp)
(5′ AG11VITCGCAACCTGGCTA 3′;
5′ TGAAGCTGCTGCTGATAGGA 3′).

PCR products were analysed on 2% agarose gels and visualised with ethidium bromide.

Results

In vivo the neural tube acquires region specific gene expression with respect to both the rostravcaudal and dorsal/ventral axes, indicative of restricted developmental fate. Expression of neural tube markers expressed in the neural tube shortly after closure in restricted anterior, posterior and ventral domains was analysed in EBM⁹, which expresses Sox1 and Sox2 but not Gbx2, equivalent to closed neural tube in vivo. The ectodermal expression patterns of the analysed genes are described in table 1. Gene expression in EBM⁹ was analysed by RT-PCR or in situ hybridisation (Gbx2 and compared to EB⁹ and EPLEB⁹, which comprise a mixed population of coils containing ectoderm and mesoderm, and a mesoderm-enriched, ectoderm deficient (International patent application PCT/AU99/00265, above) population respectively. RNA from d10 embryos was used as a positive control.

As shown in FIG. 8, the expression of genes marking presumptive forebrain (Nkx2.2), individual rhombomeres of the hindbrain (HoxB1, Krox20), midbrain/hindbrain boundary (Gbx2), posterior ectoderm and trunk (Hoxa7), and ventral neural tube (Shh) was not detected in EBM⁹. Furthermore, the absence of Shh expression that is required for specification of ventral identity in the neural tube (Echelard et al., 1993), indicates that the signalling pathways leading to ventralisation of the neural tube are not active in the EBM system.

En1, En2 and Otx1 are expressed in a broad region of the anterior neural tube around the time of closure and subsequently within defined regions of the midbrain. These genes were expressed in EBM⁹ as was Mash1, a gene expressed in domains of the neuroepithelum of the forebrain, midbrain and spinal cord between days 8.5 and 10.5 (Guillemot and Joyner, 1993). Similarly Pax3 and Pax6 were expressed in EBM⁹. While these genes are restricted positionally to dorsal and ventral aspects of the neural tube respectively, evidence from chick (Goulding et al., 1993) suggests that both these genes are expressed widely in neural tube before their expression domains become restricted in response to ventral specification.

Consistent with the described mesodermal differentiation within EPLEB, expression of neural-specific genes in these aggregates was absent or detected at very low levels. Where expression was detected it is ascribed to additional, non-neural sites of expression described in the embryo, for example, Shh expression in the prechordal plate (Marti et a., 1995), HoxB1 expression in primitive streak mesoderm (Studer et al., 1998), Hoxa7 expression in the primordia of the vertebrae and ribs (Mahon et al., 1988), Pax3 expression in newly formed somites and later in the dermomyotome (Goulding et al., 1991) and En1 expression in tissues of somitic origin (Davis and Joyner, 1988).

Gene expression was much more promiscuous in EB⁹ which expressed significant levels of all positionally restricted neural patterning genes. This indicates that stochastic differentiation within the EB system is accompanied by cryptic positional specification. Use of MEDII for pluripotent cell differentiation appears to overcome the positional specification inherent within the EB differentiation system, and as a consequence produces neural progenitor cells which are less likely to be developmentally restricted. This may be related to the lack of interaction with other cell types such as visceral endoderm and mesoderm which are not formed in the EBM system.

Conclusion

Analysis of neural tube markers with spatially restricted expression in EBM indicated that the neural progenitor cells within these aggregates have not acquired positional specification. Further, signalling systems required for positional specification are not operative. This suggests that neural progenitor cells formed from pluripotent cells in response to MEDII are unlikely to be developmentally restricted. EBM therefore provide a superior system for the generation of neural progenitors from pluripotent cells compared to chemical induction or differentiation within EB in which positionally restricted genes are expressed.

### EXAMPLE 4

### Differentiation of ES Cell-Derived Neurectoderm can be Directed to Neural Crest or Glial Lineages in Response to Exogenous Signalling

Methods

Cell culture, in situ hybridisation and immunohistochemistry were as described in example 1.

Sox 10 probes were transcribed from pSox10E.1 (obtained from Dr. Peter Koopman, IMB, Brisbane, Australia). Anti-sense and sense probes were transcribed from HindIII or BamHI cut pSox10E.1 with 17 or T3 RNA polymerase respectively.

Anti-glial fibrillary acidic protein (GFAP: Sigma #G9269) was used at a dilution of 1/1000 and detected with alkaline phosphatase conjugated goat anti-rabbit IgG (ZyMax grade, Zymed Laboratories Inc.) used at a dilution of 1/1000. Cells were blocked for 30 minutes in 10% goat serum, 2% BSA in PBS.

Neural Crest Formation

EBM⁹ were collected, washed in PBS, treated with 0.5 mM EGTA pH 7.5 for 3 minutes, washed in PBS and disaggregated to small clumps (20-200 cells) by trituration. Cell clumps were allowed to settle and single cells liberated during trituration were removed with the supernatant before plating onto tissue culture grade plasticware which had been coated with cellular fibronectin (1 μg/cm²; obtained from M. D. Bettess, Department of Biochemistry, Adelaide University, Australia) and allowed to dry. Cells were cultured in Hams F12 containing 3% FCS and 10 ng/ml FGF2 and supplemented with either 0.1% 25 μM staurosporine (Sigma) in DMSO (final concentration, 25 nM) or 0.1% DMSO. Cellular aggregates were allowed to differentiate for 48 hours before fixation in 4% PFA for 30 minutes.

Glial Lineage Formation

EBM⁹ were collected, washed in PBS and broken into small clumps as described above. Cell clumps were transferred to tissue culture plastic pretreated with poly-L-Omithine as per manufacturer's instructions (Sigma) and cultured in 50% DMEM, 50% F12, 1×ITSS, 1×N2 supplement (Sigma), 10 ng/ml FGF2, 20 ng/ml EGF (R&D Systems Inc.) and 1 μl/ml Laminin (Sigma). Medium was changed daily. After 5 days medium was changed to 50% DMEM, 50% F12, 1×ITSS, 1×N2 supplement (Sigma), 10 ng/ml FGF2 and 10 ng/ml PDGF-M (R&D Systems Inc.). Cells were fixed for analysis on day 7 or 8 of culture by treatment with 4% PFA for 30 minutes.

Results

Spontaneous differentiation of EBM leads to formation of multiple cell types including morphologically identifiable neurons and glia (data not shown). Neural tube explants from the quail have been shown to form neural crest in response to the protein kinase C inhibitor staurosporine (Newgreen & Minichiello, 1996). EBM⁹ were dissociated to clumps and cultured in medium supplemented with either 0.1% of 25 μM staurosporine in DMSO (final concentration, 25 nM) or 0.1% DMSO. Cell types produced were assessed morphologically and for expression of the mammalian neural crest marker, Sox10 (Southard-Smith et al., 1998). Within three hours of seeding into medium-containing staurosporine, EBM explants were surrounded by a halo of differentiating cells (FIG. 8A) which appeared morphologically indistinguishable from avian neural crest cells produced from avian neural tube in response to staurosporine (Newgreen & Minichiello, 1996). The phenotypic alteration induced by staurosporine was homogeneous across the population of aggregates, and was not observed in EBM explants cultured in medium containing 0.1% DMSO (FIG. 8B). This differentiation was observed in the presence of 1 nM to 100 nM staurosporine, although uniform differentiation required concentrations greater than 10 nM (data not shown). After 48 hours culture, EBM explants were analysed by in situ hybridisation for expression of Sox10 (FIG. 8C) which is up regulated on the formation of mouse neural crest in vivo (Southard-Smith et al., 1998). Sox10 expression was observed in all cells formed in response to staurosporine, but not in those cultured in medium containing 0.1% DMSO.

ES cell derived neural stem cells have been shown to differentiate to glial lineages in response to sequential culture in EGF/laminin and PDGF-AA (Brustle et al., 1999). EBM⁹ explants were cultured in medium containing FGF2 (10 ng/ml), EGF (20 ng/ml) and laminin (1 μg/ml). After 5 days EGF and laminin were omitted from the medium and PDGF-AA was added to a concentration of 10 ng/ml for a further 2-3 days. Cells were not trypsinised or triturated during differentiation. Cultures were analysed by immunohistochemistry for the expression of glial fibrillary acidic protein (GFAP), a marker expressed by both glial precursors and differentiated astrocytes (Landry et al., 1990). Differentiation of EBM⁹ explants in response to EGF/laminin and PDGF-AA follows a homogeneous morphological progression depicted in FIGS. 8E-G. >95% of differentiated cells formed from EBM explants using this protocol expressed GFAP (FIG. 8H), indicating homogeneous differentiation to cells of the glial lineage.

Conclusion

Differentiation of neurectoderm derived from pluripotent cells in response to MEDII can be directed to neural crest or glial fates by the additional of biologically relevant exogenous signalling molecules. This indicates that neurectoderm derived by differentiation of pluripotent cells in response to MEDII has differentiation properties equivalent to neurectoderm in vivo.

Homogeneous, lineage specific differentiation to both lineages was achieved. Neural crest cells are precursors in vivo to craniofacial bone and cartilage, and several different types of neurons including sensory cranial nerves, parasympathetic, sympathetic and sensory ganglia. Terminally differentiated glial cells are expected to have wide ranging biological and medical applications including the treatment of neuronal diseases using cell and gene therapy.

Unpatterned neural progenitors produced by differentiation of pluripotent cells in response to MEDII can therefore be used as a superior substrate for directed formation of ectodermal lineages of medical importance.

### EXAMPLE 5

Neural progenitors derived by differentiation of pluripotent cells in response to MEDII are capable of incorporation and differentiation in the rat brain.

Methods:

Cell Culture and Preparation

D3 ES cells expressing EGFP were formed by the transfection of D3 ES cells with pFIRES+EGFP (Example 1), and used throughout. Routine tissue culture and maintenance of ES cells was as described in Example 1. EBM were formed and cultured as described in Example 1.

For injection, EBM⁷ and EBM¹⁰ were allowed to settle and media was aspirated. Cell aggregates were washed with 10 ml PBS, and treated with 0.5 mM EGTA pH 7.5 for 3 minutes. This was removed and aggregates were treated with 3 ml of trypsin (0.05%, Gibco, UK)/EDTA (0.5 mM, Gibco, UK) for 1 minute. 1 ml of FCS (Gibco, UK) was then added and cells were dissociated by vigorous pipetting. Cells were collected by centrifugation and re-suspended to 1×10⁷ and 1×10⁸ cells/ml in Dulbecco's MEM (DMEM). The cell suspensions were chilled on ice for no more than 2 hours before implantation.

Injection Procedure

Sprague Dawley rats no older than 8 hours were chilled on ice for up to 20 minutes to reduce their metabolic rate and reduce movement. 2 μl of cell suspension or DMEM was injected into the left lateral ventricle.

A sterile 51 μl positive displacement gas chromatography syringe (SGE, UK), modified by reducing the length of the needle to 2 cm, was used for injection. Coordinates for the location of the left lateral ventricle were obtained from Paxinos et al, 1994. The needle was introduced at a 45° angle into the skull 2 mm above the left eye socket, to a depth of approximately 0.5 cm, and the cells were implanted. Successful injection into the lateral ventricle was identified by displacement of 2 μl of clear CSF from the injection site following introduction of the cells. The left rear toe was clipped for identification purposes. The newborn rats were placed under a heat lamp for 15 minutes to reset their core temperature, reunited with their mothers and observed daily.

Assessment of Cellular Incorporation

Rats were sacrificed by cervical dislocation at 1, 2, 4, 8 and 16 weeks after implantation of cells.

Brains were removed and fixed for 4 hours in 4% para-formaldehyde before dehydration. Brains were immersed in 50% ethanol for 4 hours then 70% ethanol for 4 hours. Brains were stored at 4° C. in 70% ethanol inside tissue processing cassettes (Bayer, Australia).

All brains were sectioned horizontally using a vibratome (Lancer, 1000). 0.5 mm. sections were visualised under the fluorescent (Nikon TE300, FITC, excitation 465/95 emission 515/55 nm, with the dichroic mirror set at 505 nm) or confocal (Bio/Rad MRC 1000 uv confocal system attached to a Nikon Diphot 3000 microscope) microscope. For confocal microscopy, excitation and emission bandwidths were 488/8 nm and 522/35 nm respectively and images were taken using a water immersion×40 lens with a numerical aperture of 1.15.

Sections containing green fluorescent regions were embedded in paraffin wax as follows; tissue was dehydrated through 80% ethanol for 1 hour, followed by 95% ethanol for 1.5 hours, then 100% ethanol for 4 hours. Sections were then immersed in Histoclear (Ajax, Australia)/ethanol (50%:50%) for 1 hour, then 100% Histoclear for 4 hours, followed by paraffin wax (Oxford labware, USA) at 60° C. for 2 hours and then paraffin wax under vacuum (15-20 KPa) for a further 2 hours. The 0.5 mm brain slices were removed from the molten wax bath and placed on a hot plate at 60° C. A pre-warmed mould was half filled with molten wax. The lid of the cassette was removed and tissue was positioned at the bottom of the mould and then transferred to a cold (−20° C.) surface. The mould was removed from the cold surface after the wax at the bottom solidified. The bottom of the cassette was placed on top of the base mould and tissue and the mould was filled with wax and allowed to solidify. The wax block containing tissue was separated from the mould before thin sections (7 μm, Leica RM2135 Microtome) were cut. Using fine paintbrushes, the sections were floated on the surface of a water bath heated at 45-55° C. These sections were floated onto superfrost+slides (Bayer, Australia) and allowed to dry completely prior to immunohistochemical analysis.

Immunohistochemistry.

Paraffin wax embedded 7 μm thick brain sections were washed with Histoclear twice for 4 minutes, in order to remove paraffin wax from the tissue. The slices were then treated with graded ethanol (100%, 80% and 70% each for 2 minutes) before washing with PBS twice for 5 minutes. The tissue was then permeablised with 0.1% triton-X100 (Sigma, UK), in PBS for 5 minutes and treated with blocking solution (10% goat serum, Gibco, UK, 3% bovine serum albumin, Roche, Germany) in PBS for 30 minutes at room temperature.

Antibodies for Nestin and NF200 were used as previously described in Example 1. Use of other antibodies is outlined below.

GFP: Blocking buffer containing primary antibody to GFP raised in mouse and used at 1 in 1000, Clonetec, USA. Secondary antibody: Anti-mouse IgG conjugated to alkaline phosphatase, 1 in 4000, Rockland, USA.

GFAP: Blocking buffer containing primary antibody to glial fibrilliary acidic protein, GFAP raised in rabbit used at 1 in 500, Sigma, UK; Secondary antibody: Anti-rabbit IgG conjugated to alkaline phosphatase, 1 in 500, Zymed, USA.

Primary antibodies were added to the slices overnight at 4° C. The tissue sections were then washed 3 times for 30 minutes at room temperature with PBS, and then with buffer 1 (Tris-HCl 100 mM, pH 7.5, NaCl 150 mM) for 10 minutes at room temperature. The secondary antibodies were diluted in buffer 1 containing 0.5% blocking powder (Roche, Germany) and added to the sections for 2 hours at room temperature. The tissue was then washed in buffer 1 twice for 15 minutes and then in buffer 2 (Tris-HCl 100 mM pH 9.5, NaCl 100 mM, MgCl₂ 5 mM, with 0.1% Tween 20, Sigma, UK for 10 minutes). Development solution (buffer 2 with 0.2% Nitroblue tetrazolium/5-Bromo-4-chloro-4-indolyl phosphate, Roche, Germany and 5 mM levamisole, Sigma, UK) was then added to the tissue and the dark purple colour was allowed to develop overnight in the dark.

Results

A total of 72 rats were used in the study and their treatment is summarised in table 1.

TABLE 1
Implantation of EBM⁷ and EBM¹⁰ in rat brains showing numbers of rats,
number of cells implanted, the cell type implanted or control and the time
of brain harvest.

| Number of rats | Number of cells | Cell type | Brain harvest |
|---|---|---|---|
| 8 | 200,000 | EBM⁷ | 4 at 1 week |
| | | | 4 at 2 weeks |
| 8 | 20,000 | EBM⁷ | 4 at 1 week |
| | | | 4 at 2 weeks |
| 8 | | Non injected | 4 at 1 week |
| | | | 4 at 2 weeks |
| 7 | 200,000 | EBM¹⁰ | 4 at 1 week |
| | | | 3 at 2 weeks |
| 8 | | DMEM | 4 at 1 week |
| | | | 4 at 2 weeks |
| 14 | 200,000 | EBM⁷ | 5 at 4 weeks |
| | | | 5 at 8 weeks |
| | | | 4 at 16 weeks |
| 14 | 200,000 | EBM¹⁰ | 5 at 4 weeks |
| | | | 5 at 8 weeks |
| | | | 4 at 16 weeks |
| 5 | | DMEM | 2 at 4 weeks |
| | | | 2 at 8 weeks |
| | | | 1 at 16 weeks |

Neural progenitors derived by differentiation of pluripotent cells in response to MEDII persists and disperses in the rat brain.

All the injections of the cells were performed in the absence of immunosuppression.

After one week GFP positive cells were identified in all implanted rats, predominantly located around the implantation site as multiple clumps (˜50-300 cells per clump) within the left lateral ventricle wall. Rats injected with 20,000 cells had fewer GFP positive clumps of cells compared with rats implanted with 200,000 cells. All brains harvested at 1 week showed no abnormal development when compared with the un-injected and DMEM injected control animals.

After 2 weeks, GFP positive cells were still present in the left lateral ventricle wall of all implanted rats, however additional regions of GFP positive cells in other sites such as the 3^{rd} ventricle wall and the cerebral aqueducts were identified, indicating that the cells had moved within the cerebrospinal fluid. Implanted cells at 2 weeks formed multiple clumps per brain, each clump consisting of approximately 50 to 300 GFP positive cells within the walls of the ventricular system (a typical clump is shown in FIG. 10). No apparent difference between the implantation of EBM⁷ or EBM¹⁰ cells in the brain was observed at 1 and 2 weeks.

After 4 weeks, GFP positive cells were found in all implanted brains and they had dispersed widely along the ventricle walls. Many cells had moved into the sub-ependymal layer (FIG. 11). No difference was observed between the distribution of EBM⁷ or EBM¹⁰ implants.

At 8 and 16 weeks after implantation, GFP positive cells could no longer be identified around the ventricle walls and were difficult to find. This may be due to dispersal of the cells within the brain or represent a loss of cells due to immuno rejection. In all the brains examined in both EBM⁷ and EBM¹⁰ groups at 8 and 16 weeks, GFP positive cells were identified in deeper brain regions such as the thalamus, (FIG. 12), frontal cortex, caudate putamen (FIG. 13) and colliculus, midbrain and in these sites in the un-injected right side of the brain. There were no apparent differences in the distribution of EBM⁷ and EBM¹⁰ at either 8 or 16 weeks. The distribution of implanted GFP positive cells within rat brains over 16 weeks is shown in FIG. 14. The black areas represent the location of the GFP positive cells at time points up to 4 weeks and the white at later times up to 16 weeks. The cells were initially located within the CSF, then incorporated into the ependyma and were later found in brain regions between the main ventricles and the ventricular canals.

Neural Progenitors Derived by Differentiation of Pluripotent Cells in Response to MEDII Differentiates in Rat Brain

Using confocal microscopy cellular processes (FIG. 13 arrows), were identified at 8 weeks in rats implanted with EBM⁷, which was indicative of differentiation to neurons or glia. To further assess neural differentiation, thin serial sections were taken from a GFP positive brain region for immunohistochemical analysis to assess gene expression. FIG. 15 shows serial sections from a GFP positive region located in the caudate putamen of a rat injected with EBM⁷ cells after 2 weeks. Serial sections were stained for nestin (FIG. 15A), NF200 (FIG. 15B), GFP (FIG. 15C) and GFAP (FIG. 15D). FIG. 15A shows that the GFP positive cells did not express the neural precursor nestin, however nestin was expressed in EBM (Example 1). This indicates that the implanted cells had differentiated in the rat brain. Serial sections of this GFP positive region indicated some cellular locations were immunoreactive for both GFP and the glial cell marker GFAP (FIG. 15D) and other cellular locations that were immunoreactive for both GFP and the neural marker NF200 (FIG. 15B). These data indicate that the implanted neural progenitor cells had differentiated into glia and neurons respectively.

Safety

Minimal trauma to the animals was maintained throughout the study and there was no procedure-induced mortality or infection. However, a single rat injected with 200,000 EBM⁷ cells developed a teratoma or a teratocarcinoma at one month and another had obvious hydrocephalus. The hydrocephalus was shown to be due to blockage of CSF drainage due to cell clumping around the 3^{rd} ventricle. No tumors were identified in the rats implanted with EBM¹⁰ and there was only one tumor out of 30 rats implanted with EBM⁷.

Conclusions

These data show that neural progenitors derived by differentiation of pluripotent cells in response to MEDII can incorporate, differentiate and disperse in the rat brain. The neural progenitors derived by differentiation of pluripotent cells in response to MEDII are therefore potentially of use for the replacement of damaged or dysfunctional brain cells in conditions such as Parkinson's disease, dementia, central ischaemic injury resulting from trauma or stroke, spinal injury and movement disorders.

TABLE 2
Summation of the ectodermal expression patterns of positionally
specified neural markers

| Gene | Ectodermal expression pattern in vivo | Reference |
|---|---|---|
| En1 | Detected on day 8.0 of development in a | Davis and Joyner |
| | neural folds at the level of the foregut | 1988 |
| | pocket. Expression persists at the mid- | |
| | brain/hindbrain boundary | |
| En2 | as for En1 | Davis and Joyner |
| | | 1988 |
| Gbx2 | Pan neural expression occurs prior to | Wassarman et |
| | neural tube closure (d 7.5). Expressed | al. 1997 |
| | caudal to midbrain at d9.5, and later in | |
| | the forebrain. | |
| Hoxa7 | Expressed in the posterior extoderm and | Mahon et al. |
| | the trunk | 1988 |
| HoxB1 | Expressed with Rhombomere 4 | Studer et al 1998 |
| Krox20 | Expression is established in the early | Nieto et al. 1991 |
| | neural plate (d8.0) in a single domain and | |
| | then in a second more posterior domain | |
| | (d8.5). These domains coincide with the | |
| | later position of rhombomeres 3 and 5 | |
| | respectively. | |
| Otx1 | First expressed in d8.5 embryos in a large | Simeone et al. |
| | region of the anterior neural tube. By d9- | 1993 |
| | d10, the posterior boundary of expression | |
| | coincides with the mesencephalon. | |
| Mash1 | Expressed initially in domains of the | Guillemot and |
| | neuroepithelium encompassing forebrain, | Joyner 1993 |
| | midbrain and spinal cord at 8.5 dpc. Ex- | |
| | pression is later broadened and is found | |
| | in the ventricular zone of all regions of | |
| | the brain. | |
| Nkx2.2 | Nypothalamic and thalamic regions of the | Price et al. 1992 |
| | developing forebrain. | |
| Pax3 | First expressed in 8.5 day embryos in the | Goulding et al. |
| | dorsal region of the neural groove and re- | 1991 |
| | cently closed neural tube. In the trunk | |
| | expression occurred just prior to | |
| | neural tube closure. | |
| Pax6 | First expressed at d8.0 in the forebrain and | Walther and |
| | hindbrain, and along the entire antero- | Gruss 1991 |
| | posterior axis of the spinal cord. After | |
| | neural tube closure. Expression is re- | |
| | stricted to the ventral ventricular zone. | |
| Shh | Initially detected in the ventral midbrain | Marti et al 1995 |
| | expression extends rostrally and caudally | |
| | to encompass a strip of ventral tissue | |
| | from the rostral limit of the forebrain | |
| | to the caudal regions of the spine. | |
| Sox1 | Pan specific neural marker | Pevney et al. |
| | | 1998 |
| Sox2 | Pan specific neural marker | Pevney et al. |
| | | 1998 |

### REFERENCES

Anderson D. J. (1989). The neural crest cell lineage problem: neuropoiesis? Neuron 3, 1-12.

Bain G., Kitchens D., Yao M., Huettneer J E., Gottlieb D I., (1995) Embryonic stem cells express neuronal properties in vitro. Dev Biol 168, 342-357.

Bjomson C. R., Rietze R. L., Reynolds B. A., Magli M. C. and Vescovi A. L. (1999). Turning brain into blood: a hematopoietic fate adopted by adult neural stem cells in vivo. Science; 283, 534-537.

Brustle O., Jones K. N., Learish R. D., Karram K., Choudhary K., Wiestler O. D., Duncan I. D. and McKay R. D. (1999). Embryonic stem cell-derived glial precursors: a source of myelinating transplants. Science. 285, 754-756.

Chapman, G. and Rathjen, P. D. (1995). Sequence and evolutionary conservation of the murine Gbx-2 homeobox gene. FEBS Letters 364, 289-292.

Coucouvanis, E. and Martin, G. R. (1995). Signals for death and survival: a two-step mechanism for cavitation in the vertebrate embryo. Cell 83, 279-287.

Davis, C. A. and Joyner, A. L. (1988). Expression patterns of the homeobox-containing genes En-1 and En-2 and the proto-oncogene int-1 diverge during mouse development. Genes Dev. 2, 1736-1744 Doetschman, T. C., Eistetter, H., Katz, M., Schmidt, W. and Kemler, R. (1985). The in vitro development of blastocyst derived embryonic stem cell lines: formation of visceral yolk sac, blood islands and myocardium. J. Embryol. Exp. Morph. 87, 27-45.

Echalard, Y. Epstein, D. J., St-Jacques, B., Shen, L., Mohler, J., McMahon, A. P. (1993). Sonic Hedgehog, a member of a family of putative signalling molecules, is implicated in the regulation of CNS polarity. Cell 75, 1417-1430.

Goulding, M. D., Chalepakis, G., Deutsch, U., Erselius, J. R. and Gruss, P. (1991). Pax-3, a novel murine DNA binding protein expressed during early neurogenesis. EMBO J. 10, 1135-1147.

Guillemot, F. and Joyner, A. L. (1993). Dynamic expression of the murine Achaete-Scute homologue Mash-1 in the developing nervous system. Mech. Dev. 42, 171-185.

Haub, O. and Goldfarb, M. (1991). Expression of the fibroblast growth factor-5 gene in the mouse embryo. Development 112, 397406.

Hébert, J. M., Basilico, C., Goldfarb, M., Haub, O. and Martin G. R. (1990). Isolation of cDNAs encoding four mouse FGF family members and characterisation of their expression patterns during embryogenesis. Dev. Biol. 138, 454-463.

Herrmann, B. G. (1991). Expression pattern of the Brachyury gene in whole-mount T^{Wis}/T^{Wis} mutant embryos. Development 113, 913-917.

Hogan, B., Beddington, R., Constantini, F. and Lacy, E. (1994). Manipulating the Mouse Embryo: A Laboratory Manual. pp. 330-335. Cold Spring Harbor Laboratory Press, Cold Spring Harbor, N.Y.

Kaufman, M. H. (1992). The atlas of mouse development. Academic Press Limited, London.

Lake, J., Rathjen, J., Remiszewski, J., and Rathjen, P. D., (2000). Reversible programming of pluripotent cell differentiation. J Cell Sci. 113, 555-566.

Landry, C. F., Ivy, G. O. and Brown, I. R. (1990). Developmental expression of glial fibrillary acidic protein mRNA in the rat brain analyzed by in situ hybridisation. J. Neurosci. Res. 25, 194-203.

Li, M., Pevny, L., Lovell-Badge, R. and Smith, A. (1998). Generation of neural precursors from embryonic stem cells by lineage selection. Current Biol. 8. 971-974.

Mahon, K. A., Westphal, H. and Gruss, P. (1988). Expression of the homeobox gene Hox 1.1 during mouse embryogenesis. Development 104 Suppl, 187-195.

Marti, E., Takada, R., Bumcrot, D. A., Sasaki, H. and McMahon, A. (1995). Distribution of Sonic hedgehog peptides in the developing chick and mouse embryo. Development 121, 2537-2547.

Newgreen, D. F. and Minichiello, J. (1996). Control of Epitheliomesenchymal Transformation II. Cross-Modulation of Cell Adhesion and Cytoskeletal Systems in Embryonic Neural Cells. Dev. Biol. 176, 300-312.

Nieto, M. A., Bradley, L. C. and Wilkinson, D. G. (1991). Conserved segmental expression of Krox-20 in the vertebrate hindbrain and its relationship to lineage restriction. Development Suppl. 2, 59-62.

Okabe S., Forsberg-Nilsson K., Spiro A C., Segal M., McKay R D. (1996). Development of neuronal precursor cells and functional postmitotic neurons from embryonic stem cells in vitro. Mech Dev 59, 89-102.

Paxinos, G., Ashwell, K. W. S. and Törk, I. Atlas of the developing rat nervous system. 1994; Academic press, second edition, 24 Oval Road, London, UK.

Pevny, L. H., Sockanathan, S., Placzek, M. and Lovell-Badge, R. (1998). A role for SOX1 in neural determination. Development 125, 1967-1978.

Price, M., Lazzaro, D., Pohl, T., Mattei, M. G., Ruther, U., Olivio, J. C., Duboule, D. and Lauro, R. (1992). Regional expression of the homeobox gene Nkx-2.2 in the developing mammalian forebrain. Neuron 8, 241-255.

Rathjen, J., Lake, J. -A., Bettess, M. D., Washington, J. M., Chapman, G. and Rathjen, P. D. (1999). Formation of a primitive ectoderm like cell population from ES cells in response to biologically derived factors. J. Cell Sci. 112, 601-612.

Renoncourt, Y., Carrol, P., Filippi, P., Aru, V. and Alonso, S. (1998). Neurons derived in vitro from ES cells express homeoproteins characteristic of motomeurons and interneurons. Mech. Dev. 79, 185-197.

Ronn, L. C., Hartz, B. P. and Bock, E. (1998). The neural cell adhesion molecule (NCAM) in development and plasticity of the nervous system. Exp. Gerontol. 33, 853-864.

Rosen B. and. Beddington, R. S. (1993). Whole-mount in situ hybridisation in the mouse embryo: gene expression in three dimensions. Trends Genet 9, 162-167.

Rosner, M. H., Vigano, A., Ozato, K., Timmons, P. M., Poirier, F., Rigby, P. W. J. and Staudt, L. M. (1990). A POU-domain transcription factor in early stem cells and germ cells of the mammalian embryo. Nature 345, 686-692.

Rutihauser, U. (1992). NCAM and its polysialic acid moiety: a mechanism for pull/push regulation of cell interactions during development. Development 1992 Supplement, 99-104.

Scholer, H. R., Ruppert, S., Suzuki, N., Chowdhury, K. and Gruss, P. (1990). New type of POU domain in germ line-specific protein Oct4. Nature 344, 435-439.

Shen, M. M. and Leder, P. (1992). Leukemia inhibitory factor is expressed by the preimplantation uterus and selectively blocks primitive ectoderm formation in vitro. Proc. Natl. Acad. Sci. U.S.A. 89, 8240-8244.

Simeone, A. (1998). Otx1 and Otx2 in the development and evolution of the mammalian brain. EMBO J. 17, 6790-6798.

Southarrd-Smith, E. M., Kos, L. and Pavan, W. J. (1998). Sox10 mutation disrupts neural crest development in Dom Hirschsprung mouse model. Nat. Genet 18, 60-64.

Studer, M., Gavalas, A., Marshall, H., Ariz-McNaughton, L., Rijli, F. M., Chambon, P. and Krumlauf, R. (1998). Genetic interactions between Hoxa1 and Hoxb1 reveal new roles in regulation of early hindbrain patterning. Development 125, 1025-1036.

Wassarman, K. M., Lewandoski, M., Campbell., K., Joyner, A. L., Rubenstein, J. L. R., Martinez, S. and Martin, G. R. (1997) Specification of the anterior hindbrain and establishment of a normal mid/hindbrain organizer is dependent on Gbx2 function. Development 124, 2923-2934.

Walther, C. and Gruss, P. (1991). Pax-6, a murine paired box gene, is expressed in the developing CNS. Development 113, 1435-1449.

Zimmernan, L., Parr, B., Lendahl, U., Cunningham, M., McKay, R., Gavin, B., Mann, J., Vassileva, G. and McMahon, A. (1994). Independent regulatory elements in the nestin gene direct transgene expression to neural stem cells or muscle precursors. Neuron 12, 11-24.

It will be understood that the invention disclosed and defined in this specification extends to all alternative combinations of two or more of the individual features mentioned or evident from the text or drawings. All of these different combinations constitute various alternative aspects of the invention.

It will also be understood that the term “comprises” (or its grammatical variants) as used in this specification is equivalent to the term “includes” and should not be taken as excluding the presence of other elements or features.

1. A method of producing neurectoderm cells, which method includes
providing
a source of early primitive ectoderm-like (EPL) cells;
a conditioned medium as hereinbefore defined; or an extract therefrom exhibiting neural inducing properties; and
contacting the EPL cells with the conditioned medium or extract, for a time sufficient to generate controlled differentiation to neurectoderm cells.

2. A method according to claim 1 wherein the neurectoderm cells produced are early neurectoderm cells.

3. A method according to claim 1 wherein the neurectoderm cells exhibit neural plate-like characteristics.

4. A method according to claim 2, including further providing a suitable culture medium as hereinbefore defined, and
further culturing the early neurectoderm cells in the presence of the suitable culture medium while late neurectoderm cells are formed.

5. A method according to claim 4 wherein the late neurectoderm cells so produced exhibit neural tube-like characteristics.

6. A method according to claim 1, further including the preliminary steps of
providing
a source of pluripotent cells;
a source of a biologically active factor including
a low molecular weight component selected from the group consisting of proline and peptides including proline and functionally active fragments and analogues thereof; and
a large molecular weight component selected from the group consisting of extracellular matrix portions and functionally active fragments or analogues thereof, or the low or large molecular weight component thereof;
contacting the pluripotent cells with the source of the biologically active factor, or the large or low molecular weight component thereof, to produce early primitive ectoderm-like (EPL) cells.

7. A method according to claim 6, wherein the pluripotent cells are selected from one or more of the group consisting of embryonic stem (ES) cells, in vivo or in vitro derived ICM/epiblast, in vivo or in vitro derived primitive ectoderm, primordial germ cells, EG cells, teratocarcinoma cells, EC cells, and pluripotent cells derived by dedifferentiation or by nuclear transfer.

8. A method according to claim 1, wherein the conditioned medium is MEDII.

9. A method according to claim 1, wherein the EPL cells are contacted with the neural inducing extract.

10. A method according to claim 9, wherein the neural inducing extract, excludes the biologically active factor, or the large or low molecular weight component of the conditioned medium.

11. A method according to claim 10, wherein the neural inducing extract includes a natural or synthetic molecule or molecules which compete(s) with molecules within the conditioned medium that bind to a receptor on EPL cells responsible for neural induction.

12. A method according to claim 4 wherein the further culturing step is conducted in the presence of a growth factor from the FGF family.

13. A method according to claim 1, wherein the FGF growth factor is selected from the group consisting of aFGF, bFGF and FGF4.

14. A method according to claim 13, wherein the FGF growth factor includes FGF4.

15. A method according to claim 14, wherein the FGF growth factor is present in the conditioned medium or extract in a concentration of approximately 1 to 100 ng/ml.

16. A method according to claim 4, wherein the EPL cells are cultured in the conditioned medium or extract for approximately 1 to 7 days.

17. A method according to claim 16 wherein the further culturing step is initiated on day 3.

18. A method according to claim 1, further including the step of identifying the neurectoderm cells by procedures including gene expression markers, morphology and differentiation potential.

19. A method according to claim 18, wherein the conversion of EPL cells to neurectoderm cells is characterised by
down regulation of expression of Oct4 relative to embryonic stem (ES) cells; and; and one or more of
up regulation of expression of N-Cam and nestin;
up regulation of expression of Sox1 and Sox2; and
initial up regulation of expression of Gbx2, followed by down regulation thereof as neurectoderm cells persist.

20. A method according to claim 19 wherein the upregulation of expression of Gbx2 is indicative of neurectoderm cells having neural plate-like characteristics.

21. A method according to claim 19 wherein the upregulation of expression of Gbx2 is indicative of neurectoderm cells having neural plate-like characteristics and the subsequent downregulation of Gbx2 is indicative of cells having neural tube-like characteristics.

22. A method according to claim 19 wherein the neurectoderm cells produced express neural identity genes selected from the group consisting of one or more of Otx1, Mash 1, En1, En2, Pax3 and Pax6.

23. A method according to claim 22 wherein the neurectoderm cells exhibit substantially no expression of patterning marker genes selected from the group consisting of one or more of HoxB1, Hoxa7, Krox20, Nkx2.2 and Shh.

24. A method for producing differentiated or partially differentiated cells from neurectoderm cells, which method includes
providing
neurectoderm cells produced according to claim 1;
a suitable culture medium as hereinbefore described; and optionally
a growth factor from the FGF family;
further culturing the neurectoderm cells in the cell culture medium, in the presence or absence of the FGF growth factor, to produce differentiated or partially differentiated cells.

25. A method according to claim 24, wherein the differentiated or partially differentiated cells produced are cells selected from the group consisting of neuronal cell precursors, neural crest cells, glial cell precursors, or differentiated neurons or glial cells.

26. A method according to claim 24, wherein the cell culture medium is a mixture of foetal calf serum (FCS) and Ham's F12 nutrient mixture (F12).

27. A method according to claim 25, wherein the FGF growth factor is present at a concentration in the range of approximately 1 to 100 ng/ml.

28. A method according to claim 24, wherein the additional neurectoderm cell culture step is conducted in the presence of additional growth factors and/or differentiation agents.

29. A method according to claim 28, wherein the additional neurectoderm cell culture step is conducted in the presence of a Protein Kinase Inhibitor.

30. A method according to claim 29, wherein the inhibitor is staurosporine.

31. A method according to claim 29, wherein the differentiated cells produced are substantially homogeneous populations of neural crest cells.

32. A method according to claim 28, wherein the additional neurectoderm cell culture step is conducted
in a first stage, in the presence of laminin, an FGF growth factor and an EGF growth factor; and
in a second stage, in the presence of a PDGF growth factor and in the substantial absence of EGF, FGF and laminin.

33. A method according to claim 32, wherein the differentiated cells produced are substantially homogeneous populations of glial cells.

34. A method according to claim 28, wherein the additional neurectoderm cell culture step is conducted in the presence of the conditioned medium according to claim 1.

35. A method according to claim 34, wherein the differentiated cells produced are neuronal cells in high frequency.

36. A method for maintaining neurectoderm cells in vitro in cell populations that are substantially homogeneous, which method includes
providing
neurectoderm cells produced according to claim 1; and
a suitable culture medium as hereinbefore defined;
further culturing the neurectoderm cells in the culture medium to form aggregates of neurectoderm cells.

37. A method according to claim 36, wherein the additional culturing step begins at day 3 or later.

38. A method according to claim 36 wherein the conditioned medium is a modified MedII medium wherein the foetal calf serum (FCS) is absent.

39. A neurectoderm cell derived in vitro exhibiting two of more of the following characteristics
down regulation of Oct4 expression;
substantial absence of patterning marker expression;
expression of N-CAM and nestin;
expression of Sox1 and Sox2
expression of Gbx 2;
expression of neural genes.

40. A neurectoderm cell according to claim 39, wherein the neurectoderm cell exhibits initial upregulation of Gbx2 indicative of early neurectoderm cells having neural plate-like characteristics.

41. A neurectoderm cell according to claim 0.39 wherein the neurectoderm cell exhibits initial upregulation of Gbx2, and subsequent down regulation of Gbx2 indicative of late neurectoderm cells having neural tube-like characteristics,
the late neurectoderm being further characterised by the substantial absence of patterning marker expression;
and up regulation of neural genes

42. A neurectoderm cell according to claim 39, wherein the neurectoderm cell exhibits the capacity to differentiate into all neural cell lineages including neuronal cells, glial cells and neural crest cells.

43. A neurectoderm cell according to claim 39, wherein the late neurectoderm cell exhibits substantially no expression of patterning markers selected from the group consisting of one or more of HoxB1, Hoxa7, Krox20, Nkx2.2 and Shh.

44. A neurectoderm cell according to claim 43, wherein the neurectoderm cell expresses neural identity genes selected from the group consisting of one or more of Otx1, Mash1, En1, En2, Pax3 and Pax6.

45. A neurectoderm cell according to claim 39, wherein the neurectoderm cell migrates and differentiates in vivo following brain implantation.
A neurectoderm cell according to claim 45 wherein the neurectoderm cells disperse widely along the ventricle walls and into the sub-ependymal layer, and into deeper regions of the brain, including into the uninjected side of the brain into sites that include the thalamus, frontal cortex, caudate putamen and colliculus, within the brain following intraventricular injection.

47. A neurectoderm cell according to claim 46, wherein the neurectoderm cells differentiate to form neural lineages, including neurons and glia.

48. A neurectoderm cell, or partially or terminally differentiated neurectoderm cell, whenever produced by a method according to claim 1.

49. A neurectoderm cell according- to claim 48, wherein the cell is the cell of a vertebrate selected from the group consisting of murine, human, bovine, ovine, porcine, caprine, equine and chicken.

50. A partially differentiated neuronal cell, or a terminally differentiated neuronal cell, a partially differentiated neural crest cell, or a terminally differentiated neural crest cell, a partially differentiated glial cell, or a terminally differentiated glial cell, whenever produced by a method according to any one of claims 24 to 38 or derived from neurectoderm cells according to any of claims 39 to 49.

51. Neuronal, glial or neural crest cells according to claim 50, wherein the cells are present as a substantially homogeneous population.

52. A substantially homogeneous neural crest cell population obtained in vitro exhibiting two or more of the following characteristics:
neural crest cell morphology;
cell migration; and
expression of Sox10.

53. A substantially homogeneous glial cell population obtained in vitro exhibiting one or both of the following characteristics:
glial cell morphology;
expression of the cell surface marker GFAP.

54. A substantially homogeneous glial cell population according to claim 53 wherein the cell population includes glial cell progenitors and terminally differentiated glial cells.

55. A method of producing genetically modified neurectoderm cells, which method includes
providing
a source of early primitive ectoderm-like (EPL) cells; and
a conditioned medium as hereinbefore defined; or an extract therefrom exhibiting neural inducing properties
modifying one or more genes in the EPL cells; and
contacting the genetically modified EPL cells with the conditioned medium or extract to produce genetically modified early neurectoderm cells.

56. A method according to claim 55, further including providing a suitable culture medium as hereinbefore defined, and
further culturing the early neurectoderm cells in the presence of the suitable culture medium for a time sufficient to form late neurectoderm cells.

57. A method according to claim 56, wherein the further culturing step is conducted in the presence of a growth factor from the FGF family.

58. A method of producing genetically modified neurectoderm cells, which method includes
providing
a source of genetically modified pluripotent cells;
a source of a biologically active factor including
a low molecular weight component selected from the group consisting of proline and peptides including proline and functionally active fragments and analogues thereof; and
a large molecular weight component selected from the group consisting of extracellular matrix portions and functionally active fragments or analogues thereof, or the low or large molecular weight component thereof;
a conditioned medium as hereinbefore defined; or an extract therefrom exhibiting neural inducing properties;
contacting the pluripotent cells with the source of the biologically active factor, or the large or low molecular weight component thereof, to produce genetically modified early primitive ectoderm-like (EPL) cells; and
contacting the genetically modified EPL cells with the conditioned medium or extract to produce genetically modified early neurectoderm cells.

59. A method according to claim 58, further including providing a suitable culture medium as hereinbefore defined, and
further culturing the early neurectoderm cells in the presence of the suitable culture medium for a time sufficient to form genetically modified late neurectoderm cells.

60. A method according to claim 59, wherein the further culturing step is conducted in the presence of a growth factor from the FGF family.

61. A genetically modified neurectoderm cell, a partially differentiated genetically modified neurectoderm cell, a terminally differentiated genetically modified neuronal cell, a partially differentiated genetically modified neural crest cell, or a terminally differentiated genetically modified neural crest cell, a partially differentiated genetically modified glial cell, or a terminally differentiated genetically modified glial cell produced by the methods of the present invention, whenever produced by a method according to claim 56 or 58.

62. Use of unmodified or genetically modified neurectoderm cells according to any one of claims 39 to 50 or their differentiated or partially differentiated progeny according to any one of claims 52 to 54 for use in human or animal cell therapy or transgenic animal production.

63. Use of unmodified or genetically modified neurectoderm cells according to any one of claims 39 to 50, or their differentiated or partially differentiated progeny according to any one of claims 52 to 54 for use in human or animal gene therapy.

64. Use of unmodified or genetically modified neurectoderm cells according to any one of claims 39 to 50, or their differentiated or partially differentiated progeny according to any one of claims 51 to 54 for the screening of pharmaceuticals that induce a biological response in neurectoderm cells or their differentiated or partially differentiated progeny.

65. Use of unmodified or genetically modified neurectoderm cells according to any one of claims 39 to 50, or their differentiated or partially differentiated progeny according to any one of claims 51 to 54 for the evaluation of biological molecules that direct differentiation of neural cells.

66. A method for the treatment of neuronal diseases, including Parkinson's disease, which method includes treating a patient requiring such treatment with genetically modified or unmodified neurectoderm cells according to any one of claims 39 to 50, or their partially differentiated or terminally differentiated progeny according to any one of claims 51 to 54, through human or animal cell or gene therapy.

67. A method according to claim 66, wherein the neuronal disease is Parkinson's disease, Huntington's disease, lysosymal storage diseases, multiple sclerosis, memory and behavioural disorders, or Alzheimer's disease.

68. A method for the preparation of tissue or organs for transplant, which method includes
providing neural crest cells or neurectoderm produced according to claim 51 or 52; and
culturing the neural crest cells to produce neural or non-neural cells; and the neurectoderm cells to produce neural cells.

69. A method according to claim 1, substantially as hereinbefore described with reference to any one of Examples 1 to 4.

70. Use according to claim 59, substantially as hereinbefore described with reference to Example 5.
